SEQUENCE LISTING (1 sequence)

SEQ ID NO: 1 - protein, 1615 aa, Homo sapiens
MPARTAPARVPTLAVPAISLPDDVRRRLKDLERDSLTEKECVKEKLNLLHEFLQTEIKNQLCDLETKLRKEELSEEGYLAKVKSLLNKDLSLENGAHAYNREVNGRLENGNQARSEARRVGMADANSPPKPLSKPRTPRRSKSDGEAKPEPSPSPRITRKSTRQTTITSHFAKGPAKRKPQEESERAKSDESIKEEDKDQDEKRRRVTSRERVARPLPAEEPERAKSGTRTEKEEERDEKEEKRLRSQTKEPTPKQKLKEEPDREARAGVQADEDEDGDEKDEKKHRSQPKDLAAKRRPEEKEPEKVNPQISDEKDEDEKEEKRRKTTPKEPTEKKMARAKTVMNSKTHPPKCIQCGQYLDDPDLKYGQHPPDAVDEPQMLTNEKLSIFDANESGFESYEALPQHKLTCFSVYCKHGHLCPIDTGLIEKNIELFFSGSAKPIYDDDPSLEGGVNGKNLGPINEWWITGFDGGEKALIGFSTSFAEYILMDPSPEYAPIFGLMQEKIYISKIVVEFLQSNSDSTYEDLINKIETTVPPSGLNLNRFTEDSLLRHAQFVVEQVESYDEAGDSDEQPIFLTPCMRDLIKLAGVTLGQRRAQARRQTIRHSTREKDRGPTKATTTKLVYQIFDTFFAEQIEKDDREDKENAFKRRRCGVCEVCQQPECGKCKACKDMVKFGGSGRSKQACQERRCPNMAMKEADDDEEVDDNIPEMPSPKKMHQGKKKKQNKNRISWVGEAVKTDGKKSYYKKVCIDAETLEVGDCVSVIPDDSSKPLYLARVTALWEDSSNGQMFHAHWFCAGTDTVLGATSDPLELFLVDECEDMQLSYIHSKVKVIYKAPSENWAMEGGMDPESLLEGDDGKTYFYQLWYDQDYARFESPPKTQPTEDNKFKFCVSCARLAEMRQKEIPRVLEQLEDLDSRVLYYSATKNGILYRVGDGVYLPPEAFTFNIKLSSPVKRPRKEPVDEDLYPEHYRKYSDYIKGSNLDAPEPYRIGRIKEIFCPKKSNGRPNETDIKIRVNKFYRPENTHKSTPASYHADINLLYWSDEEAVVDFKAVQGRCTVEYGEDLPECVQYSMGGPNRFYFLEAYNAKSKSFEDPPNHARSPGNKGKGKGKGKGKPKSQACEPSEPEIEIKLPKLRTLDVFSGCGGLSEGFHQAGISDTLWAIEMWDPAAQAFRLNNPGSTVFTEDCNILLKLVMAGETTNSRGQRLPQKGDVEMLCGGPPCQGFSGMNRFNSRTYSKFKNSLVVSFLSYCDYYRPRFFLLENVRNFVSFKRSMVLKLTLRCLVRMGYQCTFGVLQAGQYGVAQTRRRAIILAAAPGEKLPLFPEPLHVFAPRACQLSVVVDDKKFVSNITRLSSGPFRTITVRDTMSDLPEVRNGASALEISYNGEPQSWFQRQLRGAQYQPILRDHICKDMSALVAARMRHIPLAPGSDWRDLPNIEVRLSDGTMARKLRYTHHDRKNGRSSSGALRGVCSCVEAGKACDPAARQFNTLIPWCLPHTGNRHNHWAGLYGRLEWDGFFSTTVTNPEPMGKQGRVLHPEQHRVVSVRECARSQGFPDTYRLFGNILDKHRQVGNAVPPPLAKAIGLEIKLCMLAKARESASAKIKEEEAAKD

(19) United States
(12) Patent Application Publication
(10) Pub. No.: US 2026/0308669 A1
(43) Pub. Date: Oct. 8, 2026

(54) METHODS FOR PRESERVING METHYLATION STATUS DURING CLUSTERING

(71) Applicant: ILLUMINA, INC., San Diego, CA (US)

(72) Inventors: Colin Brown, San Diego, CA (US); Sarah E. Shutlzaberger, San Diego, CA (US)

(73) Assignee: ILLUMINA, INC., San Diego, CA (US)

(21) Appl. No.: 19/481,651

(22) PCT Filed: May 22, 2024
(86) PCT No.: PCT/US2024/030506

§ 371 Date: Nov. 5, 2025

Related U.S. Application Data
(60) Provisional application No. 63/469,160, filed on May 26, 2023.

(51) Int. Cl. C12Q 1/6844; C12Q 1/48; C12Q 1/6827; C12Q 1/6837; C12Q 1/6874; C12Q 1/6876

(52) U.S. Cl. CPC C12Q 1/6844; C12Q 1/48; C12Q 1/6827; C12Q 1/6837; C12Q 1/6874; C12Q 1/6876; C12Y 201/01037; C12Y 207/07; C12Q 2600/154

(57) ABSTRACT

The present disclosure is concerned with compositions, articles, kits, and methods for producing an array that includes clonal clusters. In one embodiment, methods include producing clonal clusters on an array that preserves methylation status, such as the methylation state of methylated CpG dinucleotides of the seed DNA molecules.

### CROSS REFERENCE TO RELATED APPLICATIONS

This application claims the benefit of U.S. Provisional Application No. 63/469,160, filed May 26, 2023, the disclosure of which is incorporated by reference herein in its entirety.

### SEQUENCE LISTING

This application contains a Sequence Listing electronically submitted to the United States Patent and Trademark Office via Patent Center as an XML file entitled “0531.002067WO01” having a size of 3,566 bytes and created on May 15, 2024. Due to the electronic filing of the Sequence Listing, the electronically submitted Sequence Listing serves as both the paper copy required by 37 CFR § 1.821(c) and the CRF required by § 1.821(e). The information contained in the Sequence Listing is incorporated by reference herein.

### FIELD

Embodiments of the present disclosure relate to preparing nucleic acids for sequencing. In particular, embodiments of the methods, compositions, systems, and kits provided herein relate to using sequencing libraries to obtain sequence data that include methylation status.

### BACKGROUND

Methylation of cytosine residues at the 5 position of the pyrimidine ring (5mC or 5meC) is proposed to have diverse roles in regulation of gene expression, parental imprinting, and molecular etiology of human diseases including cancer. The standard detection method for 5mC is whole-genome bisulfite sequencing (WGBS). This method has proven useful in obtaining whole genome methylation status; however, this method is known to cause significant degradation of sample DNA resulting in GC-bias, overestimation of 5mC abundance, and poor performance with low-input samples.

In WGBS, sodium bisulfite is used to induce deamination of unmodified cytosine residues to produce deoxyuracil, while leaving 5mC residues unaffected. When bisulfite-treated DNA is sequenced, unmodified C residues can be identified as C→T (e.g., cytosine to thymine) mutations, while 5mC residues are still read as C. This means that both non-methylated C residues and genuine T residues will be read as “T” during sequencing, in effect creating a “three-base genome” and masking C→T and T→C single nucleotide polymorphisms (SNPs). Side reactions during WGBS chemistry steps can also result in DNA backbone cleavage, leading to dropout of regions of the genome with a high proportion of nonmethylated C residues, as well as reducing overall library conversion. These issues mean that whole-genome sequencing for SNP detection cannot be performed on WGBS samples, requiring a parallel WGS library preparation and making simultaneous detection of SNPs and methylation impossible for low-input samples.

### SUMMARY OF THE APPLICATION

Sensitive detection of methylation signals by whole-genome bisulfite sequencing (WGBS) is aided by amplification of bisulfite-converted sequences by standard 4-base PCR following conversion. Many problems with degradation and dropout could in principle be mitigated by performing an amplification step prior to bisulfite conversion, but standard PCR chemistry does not allow for propagation of the methylation signal during amplification. Prior work has attempted to create “methylation-aware” PCR by including the human maintenance methyltransferase in the reaction. In one embodiment, a template-dependent DNA methyltransferase, such as the enzyme DNA (cytosine-5)-methyltransferase 1 (DNMT1), preferentially identifies hemi-methylated CpG dinucleotides sites. A hemi-methylated CpG dinucleotide, also referred to as a hemi-methylated site, describes a situation where a cytosine of a CpG dinucleotide is methylated on one strand but the cytosine of the complementary CpG dinucleotide on the other strand is not methylated. DNMT1 methylates the cytosine of the complementary CpG dinucleotide, converting the hemi-methylated site to CpG dinucleotides on both strands. Thus, a template-dependent DNA methyltransferase, such as DNMT1 allows propagation of the methylation signal at CpG sites following the extension step of PCR. However, wild-type DNMT1 is unable to survive the high temperatures encountered during PCR cycling. For this reason, DNMT1-based methyl-CpG amplification requires either the use of an engineered thermostable DNMT1 or the addition of fresh DNMT1 following each PCR cycle.

Presented herein is an alternative CpG amplification strategy that extends existing methods for creating clonal clusters in an array, such as flow cell nanowells, to allow preservation of the CpG methylation state of a seed molecule across the entire cluster. This enables an on-array workflow for bisulfite sequencing that incorporates a CpG amplification step prior to bisulfite treatment, with the potential to avoid issues with sequence bias and DNA damage as well as allow for ‘pseudo-four-base’ bisulfite sequencing.

### Definitions

Terms used herein will be understood to take on their ordinary meaning in the relevant art unless specified otherwise. Several terms used herein and their meanings are set forth below.

As used herein, the term “array” refers to a population of sites that can be differentiated from each other according to relative location. Different molecules that are at different sites of an array can be differentiated from each other according to the locations of the sites in the array. An individual site of an array can include one or more molecules of a particular type. For example, a site can include a single target nucleic acid molecule having a particular sequence or a site can include several nucleic acid molecules having the same sequence (and/or complementary sequence thereof). The sites of an array can be different features located on the same substrate. Exemplary features include without limitation, wells in a substrate, beads (or other particles) in or on a substrate, projections from a substrate, ridges on a substrate or channels in a substrate. The sites of an array can be separate substrates each bearing a different molecule. Different molecules attached to separate substrates can be identified according to the locations of the substrates on a surface to which the substrates are associated or according to the locations of the substrates in a liquid or gel. Exemplary arrays in which separate substrates are located on a surface include, without limitation, those having beads in wells.

As used herein, the term “amplification site” refers to a site in or on an array where one or more amplicons can be generated. An amplification site can be further configured to provide for immobilizing or attaching a nucleic acid at the site and to contain, hold, or attach at least one amplicon that is generated at the site.

As used herein, the term “amplicon,” when used in reference to a nucleic acid, means the product of copying the nucleic acid, wherein the product has a nucleotide sequence that is the same as or complementary to at least a portion of the nucleotide sequence of the nucleic acid. An amplicon can be produced by any of a variety of amplification methods that use the nucleic acid, e.g., a target nucleic acid or an amplicon thereof, as a template including, for example, polymerase extension, polymerase chain reaction (PCR), rolling circle amplification (RCA), ligation extension, isothermal amplification (e.g., kinetic exclusion amplification), or ligation chain reaction. An amplicon can be a nucleic acid molecule having a single copy of a particular nucleotide sequence (e.g., a polymerase extension product) or multiple copies of the nucleotide sequence (e.g., a concatameric product of RCA). A first amplicon of a target nucleic acid is typically a complementary copy. Subsequent amplicons are copies that are created, after generation of the first amplicon, from the target nucleic acid or from the first amplicon. A subsequent amplicon can have a sequence that is substantially complementary to the target nucleic acid or substantially identical to the target nucleic acid. In one embodiment where the amplification occurs at amplification sites of an array, the first amplicon is produced using a template strand, where the template strand is obtained from a sample and is not subject to amplification prior to annealing the template strand to the amplification site (see “seed nucleic acid” herein). As described in detail herein, the addition of template strands to amplification sites permits the retention of methylated nucleotides, including methylated cytosines of CpG dinucleotides.

As used herein, the term “interstitial region” refers to an area in a substrate or on a surface that separates other areas of the substrate or surface. For example, an interstitial region can separate one feature of an array from another feature of the array. The two regions that are separated from each other can be discrete, lacking contact with each other. In another example, an interstitial region can separate a first portion of a feature from a second portion of a feature. The separation provided by an interstitial region can be partial or full separation. Interstitial regions will typically have a surface material that differs from the surface material of the features on the surface. For example, features of an array can have an amount or concentration of capture agents that exceeds the amount or concentration present at the interstitial regions. In some embodiments the capture agents may not be present at the interstitial regions.

As used herein, the term “capture agent” refers to a material, chemical, molecule, or moiety thereof that is capable of attaching, retaining, or binding to a target molecule (e.g., a target nucleic acid). Exemplary capture agents include, without limitation, a capture nucleic acid that is complementary to at least a portion of a modified target nucleic acid (e.g., a universal capture binding sequence), a member of a receptor-ligand binding pair (e.g., avidin, streptavidin, biotin, lectin, carbohydrate, nucleic acid binding protein, epitope, antibody, etc.) capable of binding to a modified target nucleic acid (or linking moiety attached thereto), or a chemical reagent capable of forming a covalent bond with a modified target nucleic acid (or linking moiety attached thereto). In one embodiment, a capture agent is a nucleic acid. A nucleic acid capture agent can also be used as an amplification primer.

The terms “P5” and “P7” may be used when referring to a nucleic acid capture agent. The terms “P5′” (P5 prime) and “P7′” (P7 prime) refer to the complement of P5 and P7, respectively. It will be understood that any suitable nucleic acid capture agent can be used in the methods presented herein, and that the use of P5 and P7 are exemplary embodiments only. Uses of nucleic acid capture agents such as P5 and P7 on flow cells is known in the art, as exemplified by the disclosures of WO 2007/010251, WO 2006/064199, WO 2005/065814, WO 2015/106941, WO 1998/044151, and WO 2000/018957. One of skill in the art will recognize that a nucleic acid capture agent can also function as an amplification primer. For example, any suitable nucleic acid capture agent can act as a forward amplification primer, whether immobilized or in solution, and can be useful in the methods presented herein for hybridization to a sequence (e.g., a universal capture binding sequence) and amplification of a sequence. Similarly, any suitable nucleic acid capture agent can act as a reverse amplification primer, whether immobilized or in solution, and can be useful in the methods presented herein for hybridization to a sequence (e.g., a universal capture binding sequence) and amplification of a sequence. In view of the general knowledge available and the teachings of the present disclosure, one of skill in the art will understand how to design and use sequences that are suitable for capture and amplification of target nucleic acids as presented herein.

As used herein, the term “universal sequence” refers to a region of sequence that is common to two or more target nucleic acids, where the molecules also have regions of sequence that differ from each other. A universal sequence that is present in different members of a collection of molecules can allow capture of multiple different nucleic acids using a population of capture nucleic acids that are complementary to a portion of the universal sequence, e.g., a universal capture binding sequence. Non-limiting examples of universal capture binding sequences include sequences that are identical to or complementary to P5 and P7 primers. Similarly, a universal sequence present in different members of a collection of molecules can allow the replication or amplification of multiple different nucleic acids using a population of universal primers that are complementary to a portion of the universal sequence, e.g., a universal primer binding site. Target nucleic acid molecules may be modified to attach universal adapters (also referred to herein as adapters), for example, at one or both ends of the different target sequences, as described herein.

As used herein, the term “adapter” and its derivatives, e.g., universal adapter, refers generally to any linear oligonucleotide which can be attached to a target nucleic acid. In some embodiments, the adapter is substantially non-complementary to the 3′ end or the 5′ end of any target sequence present in a sample. In some embodiments, suitable adapter lengths are in the range of about 10-100 nucleotides, about 12-60 nucleotides and about 15-50 nucleotides in length. Generally, the adapter can include any combination of nucleotides and/or nucleic acids. In some aspects, the adapter can include one or more cleavable groups at one or more locations. In another aspect, the adapter can include a sequence that is substantially identical, or substantially complementary, to at least a portion of a primer, for example a capture nucleic acid. In some embodiments, the adapter can include a barcode, also referred to as an index or tag, to assist with downstream error correction, identification, or sequencing. The terms “adaptor” and “adapter” are used interchangeably.

As used herein, the term “nucleic acid” is intended to be consistent with its use in the art and includes naturally occurring nucleic acids and functional analogs thereof. Particularly useful functional analogs are capable of hybridizing to a nucleic acid in a sequence specific fashion or capable of being used as a template for replication of a particular nucleotide sequence. Naturally occurring nucleic acids generally have a backbone containing phosphodiester bonds. An analog structure can have an alternate backbone linkage including any of a variety of those known in the art. Naturally occurring nucleic acids generally have a deoxyribose sugar (e.g., found in deoxyribonucleic acid (DNA)) or a ribose sugar (e.g., found in ribonucleic acid (RNA)). A nucleic acid can contain any of a variety of analogs of these sugar moieties that are known in the art. A nucleic acid can include native or non-native bases. In this regard, a native deoxyribonucleic acid can have one or more bases selected from adenine, thymine, cytosine or guanine and a ribonucleic acid can have one or more bases selected from uracil, adenine, cytosine or guanine. Useful non-native bases that can be included in a nucleic acid are known in the art.

As used herein, a “seed nucleic acid” is a member of a sequencing library that has been exposed to conditions that preserve an epigenetic marker present, such as the methylation state of nucleotides. For instance, a sequencing library is seed nucleic acids if the sequencing library is not exposed to conditions resulting in linear or exponential amplification. As described herein, a seed nucleic acid can be the single-stranded nucleic acid that is annealed to a capture nucleic acid at the surface of an amplification site and used as a template strand for production of an amplicon. Also as described herein, a seed nucleic acid can be the single-stranded nucleic acid that is immobilized to an amplification site at its 5′ end and has been exposed to conditions to propagate an epigenetic marker present, such as the methylation state of nucleotides. The term “target,” when used in reference to a nucleic acid, refers to nucleic acid molecules where identification of its nucleotide sequence is desired. Unless described otherwise herein, a target nucleic acid can be a seed nucleic acid or an amplicon. A target nucleic acid having a universal sequence at each end, for instance a universal adapter at each end, can be referred to as a “modified target nucleic acid.” The terms “sequencing library” and “library” refer to the collection of target nucleic acids or modified target nucleic acids.

As used herein, the terms “clonal cluster,” “clonal population” and “monoclonal population” are used interchangeably and refer to a population of nucleic acids that is homogeneous with respect to a particular nucleotide sequence. The homogenous sequence is typically at least 10 nucleotides long, but can be even longer including for example, at least 50, at least 100, at least 250, at least 500, or at least 1000 nucleotides long. A clonal population can be derived from a single target nucleic acid. Typically, all of the nucleic acids in a clonal population will have the same nucleotide sequence. It will be understood that a small number of mutations (e.g., due to amplification artifacts) can occur in a clonal population without departing from clonality. It will also be understood that a small number of different target nucleic acid (e.g., due to a target nucleic acid that was not amplified or amplified to a limited degree) can occur in a clonal population without departing from clonality.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

It is understood that wherever embodiments are described herein with the language “include,” “includes,” or “including,” and the like, otherwise analogous embodiments described in terms of “consisting of” and/or “consisting essentially of” are also provided. The term “consisting of” means including, and limited to, whatever follows the phrase “consisting of.” That is, “consisting of” indicates that the listed elements are required or mandatory, and that no other elements may be present. The term “consisting essentially of” indicates that any elements listed after the phrase are included, and that other elements than those listed may be included provided that those elements do not interfere with or contribute to the activity or action specified in the disclosure for the listed elements.

Unless otherwise specified, “a,” “an,” “the,” and “at least one” are used interchangeably and mean one or more than one.

Conditions that are “suitable” for an event to occur or “suitable” conditions are conditions that do not prevent such events from occurring. Thus, these conditions permit, enhance, facilitate, and/or are conducive to the event.

As used herein, “providing” in the context of an item described herein, such as a composition, an article, or a nucleic acid means making the composition, article, or nucleic acid, purchasing the composition, article, or nucleic acid, or otherwise obtaining the compound, composition, article, or nucleic acid.

Also herein, the recitations of numerical ranges by endpoints include all numbers subsumed within that range (e.g., 1 to 5 includes 1, 1.5, 2, 2.75, 3, 3.80, 4, 5, etc.).

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible Subranges as well as individual numerical values within that range. For example, description of a range such as from 1 to 6 should be considered to have specifically disclosed Subranges Such as from 1 to 3, from 1 to 4, from 1 to 5, from 2 to 4, from 2 to 6, from 3 to 6 etc., as well as individual numbers within that range, for example, 1, 2, 2.7.3, 4, 5, 5.3, and 6. This applies regardless of the breadth of the range.

For any method disclosed herein that includes discrete steps, the steps may be conducted in any feasible order. And, as appropriate, any combination of two or more steps may be conducted simultaneously.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The following detailed description of illustrative embodiments of the present disclosure may be best understood when read in conjunction with the following drawings.

FIG. 1A-1I shows schematic drawings of an embodiment of producing clonal clusters that preserve the CpG methylation state of a seed nucleic acid. For simplicity, only one amplification site of an array and a limited number of target nucleic acids are shown. The figures use the following convention when numbering single strands of nucleic acids: strands showing a methylated cytosine are numbered (e.g., strand 13 of FIG. 1C); strands showing a non-methylated cytosine are also numbered but the number is modified with the symbol “‘” (e.g., strand 13’ of FIG. 1B); and strands treated with sodium bisulfite to convert non-methylated cytosines to uracils and their complement are also numbered but the number is modified with the symbol “ ” “(e.g., strand 13” of FIG. 1H and its complement 12″ of FIG. 1I).

FIG. 2 shows a schematic drawing of an alternate embodiment of producing clonal clusters that preserve the CpG methylation state of a seed nucleic acid. For simplicity, only one amplification site of an array and a limited number of target nucleic acids are shown. In this embodiment, treatment with DNMT1 occurs during bridge amplification. This embodiment of bridge amplification utilizes thermocycling amplification.

FIG. 3A-3G shows a schematic drawing of an alternate embodiment of producing clonal clusters that preserve the CpG methylation state of a seed nucleic acid. For simplicity, only one amplification site of an array and a limited number of target nucleic acids are shown. In this embodiment, treatment with DNMT1 occurs simultaneously with kinetic exclusion bridge amplification.

FIG. 4 shows the sequence of the human template-dependent DNA (cytosine-5)-methyltransferase 1 (DNMT1, SEQ ID NO:1).

The schematic drawings are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar to other numbered components.

### DETAILED DESCRIPTION

Presented herein are methods and compositions related to sequencing nucleic acids. The present disclosure provides methods for determining the methylation status of genomic DNA by producing clonal clusters that preserve epigenetic markers, such as the methylation state of a seed nucleic acid.

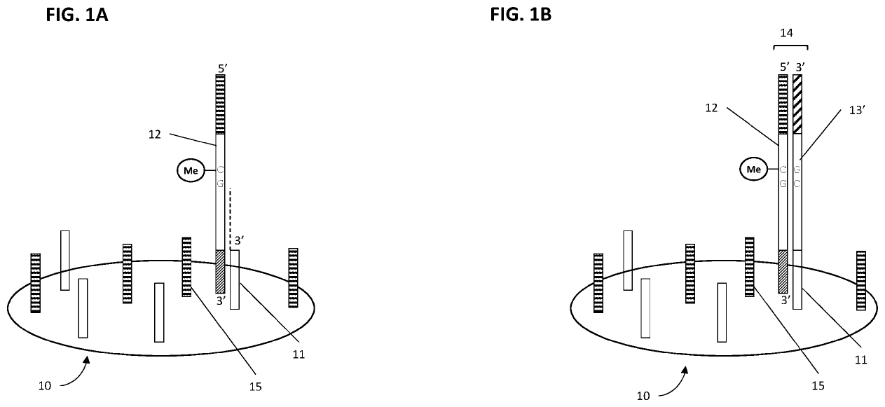

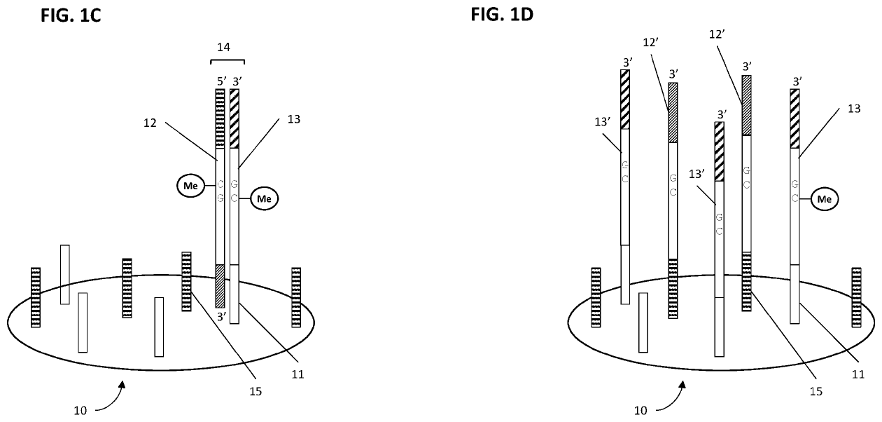

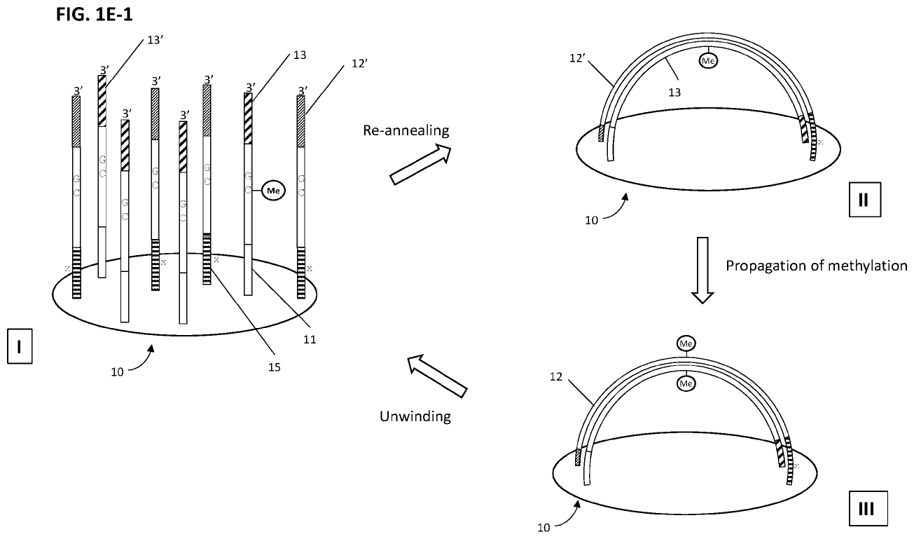

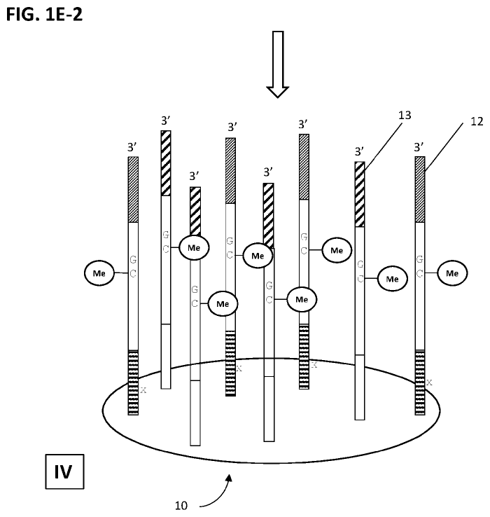

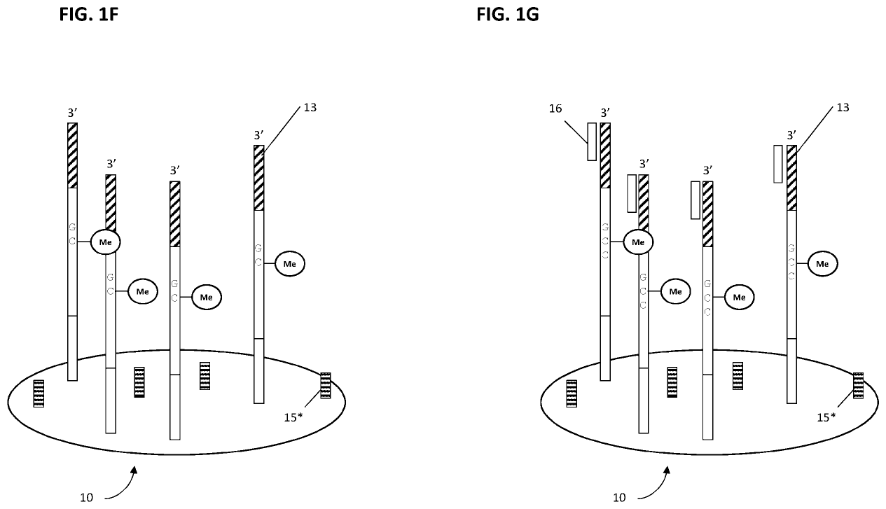

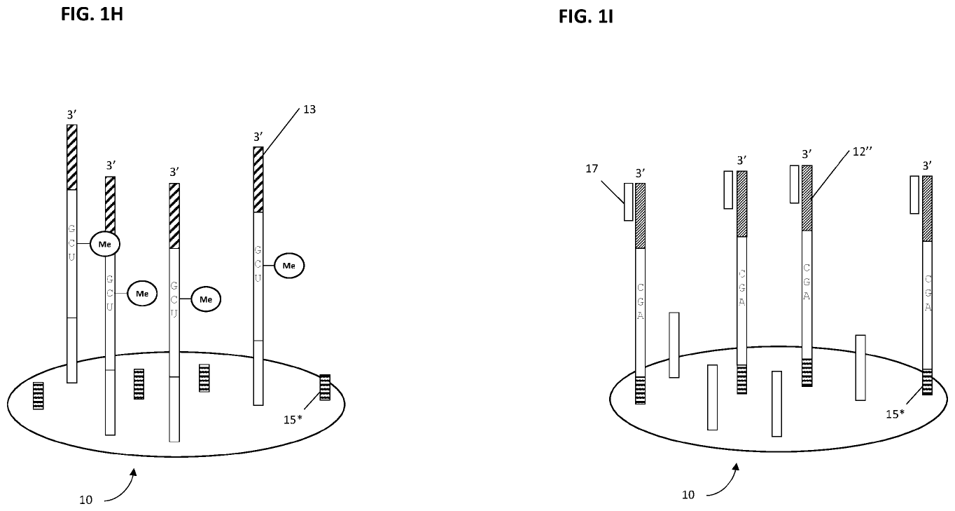

In one embodiment, the method includes providing an array that includes a plurality of amplification sites. Each amplification site includes a plurality of capture nucleic acids attached by the 5′ end to the amplification site. Individual amplification sites can include one single-stranded (ss) nucleic acid attached to the amplification site surface by hybridization between nucleotides at the 3′ end of the single-strand nucleic acid and nucleotides at the 3′ end of one capture nucleic acid. For instance, as shown in FIG. 1A the single-strand nucleic acid 12 is shown annealed to a capture nucleic acid 11. The single-strand nucleic acid is a seed nucleic acid; it is a member of a sequencing library that has been exposed to conditions that preserve an epigenetic marker present, such as the methylation state of nucleotides. In one embodiment, the sequencing library has been produced using methods that do not include amplification. For instance, in FIG. 1A the single-strand nucleic acid 12 is shown with a CpG dinucleotide where the C is methylated. The skilled person will recognize that a single-strand nucleic acid could include any number of methylated nucleotides, including multiple distinct CpG dinucleotides where the C is methylated.

The method further includes extension of the 3′ end of the capture nucleic acid with a DNA polymerase using the single-strand nucleic acid as template strand to produce the complementary strand. For example, as shown in FIG. 1B a double-stranded (ds) nucleic acid 14 results after the extension, where one strand made up of the capture nucleic acid 11 and the newly synthesized amplicon 13′, which is the unmethylated complement of the template strand 12. The complementary methylated CpG dinucleotide of strand 12 is present on the complementary strand 13′, but the methylation status is not preserved, resulting in a hemi-methylated state for that dinucleotide present on the double-stranded nucleic acid 14.

The method includes exposing the array to conditions that transfer the hemi-methylated CpG dinucleotides of the original single-strand nucleic acid to the complementary strand. As used herein, when a methylation status is “transferred” from a hemimethylated CpG dinucleotide to the complementary strand, the resulting product is a CpG dinucleotide wherein both Cs are methylated. In one embodiment, the conditions include exposing the amplification sites to an enzyme, such as DNMT1. For instance, as shown in FIG. 1C, the methylation state of the methylated CpG dinucleotide of the template strand 12 is transferred to the now methylated complementary strand 13, converting the hemi-methylated site to methylated CpG dinucleotides on both strands. It should be apparent to one skilled in the art that such a treatment would not result in methylation at CpG sites that were not originally hemimethylated, e.g., a nonmethylated CpG site would remain unmethylated after treatment.

Following transfer of the methylation status of the methylated CpG dinucleotides to the complementary strand at amplification sites, the single double-stranded nucleic acid at each amplification site is amplified to include a clonal population of immobilized nucleic acids. The clonal population includes a first sub-population of single-strand nucleic acids having the same nucleotide sequence as the complement described above (e.g., complementary strand 13′), but the CpG dinucleotides are not methylated. One of the strands of this sub-population includes the methylated CpG dinucleotides produced by the transfer using, for instance, the enzyme DNMT1. The clonal population also includes a second sub-population of single-stranded nucleic acids which includes the nucleotide sequence of the template strand, but do not contain any methylated CpG dinucleotides. For instance, as shown in FIG. 1D, an amplification site includes one methylated strand 13 and multiple copies of the same unmethylated nucleotide sequence 13′. The amplification sites also include multiple copies of the unmethylated nucleotide sequence 12′, which includes the same nucleotide sequence as 12 in FIG. 1A-C but is now attached to the amplification site surface by a different capture nucleic acid 15.

The method further includes propagating, at each amplification site, the methylated CpG dinucleotide present on one strand of the clonal population to other members of the clonal population. In one embodiment, an isothermal amplification reaction can be performed by incubating the amplification sites with a reaction mixture under conditions that transfer the methylated CpG dinucleotides of one strand of the clonal population to other strands. Thus, instead of amplifying nucleic acids, the conditions propagate the methylation status to both template and complementary strands. In one embodiment, the conditions include exposing the amplification sites to an enzyme such as DNMT1 and a DNA helicase or recombinase. Propagation of the CpG signal occurs by many cycles of steps that include (i) hybridization of the complementary single-strand nucleic acids within each amplification site, forming a bridged double-stranded fragment, (ii) transfer of any methylated C of methylated CpG dinucleotides from one strand to the other paired strand by an enzyme such as DNMT1, and (iii) unwinding of the now fully-methylated duplex by either helicase-mediated unwinding or strand invasion by another fragment in the cluster mediated by a recombinase. For instance, as shown in FIG. 1E, complementary strands 12′ and 13 shown in I anneal to form the double-stranded hemi-methylated structure shown in II. The methylation status of CpG dinucleotides on strand 13 is transferred to strand 12′ to convert strand 12′ to 12 as shown in III. The double-stranded structure shown in III is unwound and the process is repeated until the methylation status of the one methylated single-stranded nucleic acid 13 is propagated through both sub-populations of nucleic acids at the amplification sites, resulting in fully methylated clusters IV.

To facilitate sequencing of one of the sub-populations, one of the capture nucleic acids that is attached to the surface is cleaved to remove one sub-population of single-stranded nucleic acids from the amplification sites. The cleaving of a nucleotide sequence to permit the optional removal of a specific strand is referred to herein as “linearization.” For instance, as shown in FIG. 1F, the sub-population of amplicons 12 have been removed, leaving the amplicons 13 ready for sequencing. The method further includes hybridizing a sequencing primer to the single-stranded amplicon and using standard sequencing methods, such as sequencing-by-synthesis (SBS), to generate a reference read representing the full 4-base genomic sequence of the target nucleic acid. For instance, as shown in FIG. 1G a sequencing primer 16 annealed to the single-stranded amplicon 13.

After removal of the complementary strand produced during the sequencing to re-generate the single-stranded amplicons 13, the amplification sites of the array are exposed to conditions for on-array chemical treatment to allow detection of methylated residues. For instance, as shown in FIG. 1H, treatment of the array with sodium bisulfite converts all non-methylated cytosines to uracils to result in methylated and chemically treated strand 13″.

The converted amplicons can be used in a standard paired-end resynthesis and linearization to generate amplification sites complementary to the reference read, where the G→A mutations show the locations of the non-methylated cytosines and the guanosine residues show the locations of methylated cytosines.

For instance, as shown in FIG. 1I, the sub-population of amplicons 13″ have been removed after being used to synthesize amplicons 12″. FIG. 1I also shows a sequencing primer 17 annealed to the single-stranded amplicon 12″.

### Arrays

An array of amplification sites used in a method set forth herein can be present as one or more substrates. Exemplary types of substrate materials that can be used for an array include glass, modified glass, functionalized glass, inorganic glasses, microspheres (e.g., inert and/or magnetic particles), plastics, polysaccharides, nylon, nitrocellulose, ceramics, resins, silica, silica-based materials, carbon, metals, an optical fiber or optical fiber bundles, polymers and multiwell (e.g., microtiter) plates. Exemplary plastics include acrylics, polystyrene, copolymers of styrene and other materials, polypropylene, polyethylene, polybutylene, polyurethanes and Teflon™ Exemplary silica-based materials include silicon and various forms of modified silicon.

In particular embodiments, a substrate can be within or part of a vessel such as a well, tube, channel, cuvette, Petri plate, bottle, or the like. A particularly useful vessel is a flow-cell, for example, as described in U.S. Pat. No. 8,241,573 or Bentley et al., Nature 456:53-59 (2008). Exemplary flow-cells are those that are commercially available from Illumina, Inc. (San Diego, Calif.). Another particularly useful vessel is a well in a multi-well plate or microtiter plate.

In some embodiments, the amplification sites of an array can be configured as features on a surface. The features can be present in any of a variety of desired formats. For example, the sites can be wells, pits, channels, ridges, raised regions, pegs, posts or the like. In one embodiment, the amplification sites can contain beads. However, in particular embodiments the sites need not contain a bead or particle. Exemplary sites include wells that are present in substrates used for commercially available sequencing platforms (e.g., Ion Torrent, (a subsidiary of Thermo Fisher Scientific). Other substrates having wells include, for example, etched fiber optics and other substrates described in U.S. Pat. Nos. 6,266,459; 6,355,431; 6,770,441; 6,859,570; 6,210,891; 6,258,568; 6,274,320; 8,262,900; 7,948,015; U.S. Pat. Pub. No. 2010/0137143; U.S. Pat. No. 8,349,167, or PCT Publication No. WO 00/63437. In several cases the substrates are exemplified in these references for applications that use beads in the wells. The well-containing substrates can be used with or without beads in the methods or compositions of the present disclosure. In some embodiments, wells of a substrate can include gel material (with or without beads) as set forth in U.S. Pat. No. 9,512,422.

The amplification sites of an array can be metal features on a non-metallic surface such as glass, plastic or other materials exemplified herein. A metal layer can be deposited on a surface using methods known in the art such as wet plasma etching, dry plasma etching, atomic layer deposition, ion beam etching, chemical vapor deposition, vacuum sputtering, or the like. Any of a variety of commercial instruments can be used as appropriate including, for example, the FlexAL®, OpAL®, Ionfab 300Plus®, or Optofab 3000® systems (Oxford Instruments, UK). A metal layer can also be deposited by e-beam evaporation or sputtering as set forth in Thornton, Ann. Rev. Mater. Sci. 7:239-60 (1977). Metal layer deposition techniques can be combined with photolithography techniques to create metal regions or patches on a surface. Exemplary methods for combining metal layer deposition techniques and photolithography techniques are provided in U.S. Pat. Nos. 8,778,848 and 8,895,249.

An array of features can appear as a grid of spots or patches. The features can be located in a repeating pattern or in an irregular non-repeating pattern. Particularly useful patterns are hexagonal patterns, rectilinear patterns, grid patterns, patterns having reflective symmetry, patterns having rotational symmetry, or the like. Asymmetric patterns can also be useful. The pitch can be the same between different pairs of nearest neighbor features or the pitch can vary between different pairs of nearest neighbor features. In particular embodiments, features of an array can each have an area that is larger than about 100 nm², 250 nm², 500 nm², 1 μm², 2.5 μm², 5 μm², 10 μm², 100 μm², or 500 μm². Alternatively or additionally, features of an array can each have an area that is smaller than about 1 mm², 500 μm², 100 μm², 25 μm², 10 μm², 5 μm², 1 μm², 500 nm², or 100 nm². Indeed, a region can have a size that is in a range between an upper and lower limit selected from those exemplified above.

For embodiments that include an array of features on a surface, the features can be discrete, being separated by interstitial regions. The size of the features and/or spacing between the regions can vary such that arrays can be high density, medium density, or lower density. High density arrays are characterized as having regions separated by less than about 15 μm. Medium density arrays have regions separated by about 15 to 30 μm, while low density arrays have regions separated by greater than 30 μm. An array useful in the disclosure can have regions that are separated by less than 100 μm, 50 μm, 10 μm, 5 μm, 1 μm, or 0.5 μm.

In particular embodiments, an array can include a collection of beads or other particles. The particles can be suspended in a solution or they can be located on the surface of a substrate. Examples of bead arrays in solution are those commercialized by Luminex (Austin, TX, USA). Examples of arrays having beads located on a surface include those wherein beads are located in wells such as a BeadChip array (Illumina Inc., San Diego, CA, USA) or substrates used in sequencing platforms from Ion Torrent (a subsidiary of Life Technologies, Carlsbad, CA USA). Other arrays having beads located on a surface are described in U.S. Pat. Nos. 6,266,459; 6,355,431; 6,770,441; 6,859,570; 6,210,891; 6,258,568; 6,274,320; U.S. Pat. Pub. No. 2009/0026082 A1; U.S. Pat. Pub. No. 2009/0127589 A1; U.S. Pat. Pub. No. 2010/0137143 A1; U.S. Pat. Pub. No. 2010/0282617 A1; or PCT Publication No. WO 00/63437. Several of the above references describe methods for attaching target nucleic acids to beads prior to loading the beads in or on an array substrate. It will be understood, however, that the beads can be made to include amplification primers and the beads can then be used to load an array, thereby forming amplification sites for use in a method set forth herein. As set forth herein, the substrates can be used without beads. For example, amplification primers can be attached directly to the wells or to gel material in wells. Thus, the references are illustrative of materials, compositions or apparatus that can be modified for use in the methods and compositions set forth herein.

Amplification sites of an array can include a plurality of capture agents capable of binding to target nucleic acids. In one embodiment, a capture agent includes a capture nucleic acid. The nucleotide sequence of the capture nucleic acid is complementary to a universal sequence of the target nucleic acids. In some embodiments, the capture nucleic acid can also function as a primer for amplification of the target nucleic acid. In some embodiments, one population of capture nucleic acid includes a P5 primer or the complement thereof. In some embodiments, the amplification sites also include a plurality of a second capture nucleic acid, and this second capture nucleic acid can include a P7 primer or the complement thereof. In some embodiments a capture nucleic acid can include a cleavage site. Cleavage sites in a capture nucleic acid are described in greater detail herein.

In particular embodiments, a capture agent, such as a capture nucleic acid, can be attached to the amplification sites. For example, the capture agent can be attached to the surface of a feature of an array. The attachment can be via an intermediate structure such as a bead, particle or gel. An example of attachment of capture nucleic acids to an array via a gel is described in U.S. Pat. No. 8,895,249 and further exemplified by flow cells available commercially from Illumina Inc. (San Diego, CA, USA) or described in WO 2008/093098. Exemplary gels that can be used in the methods and apparatus set forth herein include, but are not limited to, those having a colloidal structure, such as agarose; polymer mesh structure, such as gelatin; or cross-linked polymer structure, such as polyacrylamide, SFA (see, for example, US Pat. App. Pub. No. 2011/0059865 A1) or PAZAM (see, for example, U.S. Prov. Pat. App. Ser. No. 61/753,833 and U.S. Pat. No. 9,012,022). Attachment via a bead can be achieved as exemplified in the description and cited references set forth previously herein.

In some embodiments, the features on the surface of an array substrate are non-contiguous, being separated by interstitial regions of the surface. Interstitial regions that have a substantially lower quantity or concentration of capture agents, compared to the features of the array, are advantageous. Interstitial regions that lack capture agents are particularly advantageous. For example, a relatively small amount or absence of capture moieties at the interstitial regions favors localization of target nucleic acids, and subsequently generated clusters, to desired features. In particular embodiments, the features can be concave features in a surface (e.g., wells) and the features can contain a gel material. The gel-containing features can be separated from each other by interstitial regions on the surface where the gel is substantially absent or, if present the gel is substantially incapable of supporting localization of nucleic acids. Methods and compositions for making and using substrates having gel containing features, such as wells, are set forth in U.S. Prov. App. No. 61/769,289.

### Target Nucleic Acids

An array used in a method described herein includes modified target nucleic acids. The target nucleic acid may be essentially any nucleic acid of known or unknown sequence. It may be, for example, a fragment of genomic DNA. Sequencing may result in determination of the sequence of the whole or a part of the target molecule. In one embodiment, the targets can be processed into templates suitable for amplification by the placement of universal amplification sequences, e.g., sequences present in a universal adaptor, at the ends of each target fragment.

The primary nucleic acid sample may originate in double-stranded DNA (dsDNA) form (e.g., genomic DNA or genomic DNA fragments). The precise sequence of the polynucleotide molecules from a primary nucleic acid sample is generally not material to the disclosure and may be known or unknown.

In one embodiment, the primary polynucleotide molecules from a primary nucleic acid sample are DNA molecules. More particularly, the primary polynucleotide molecules represent the entire genetic complement of an organism and are genomic DNA molecules which include both intron and exon sequences, as well as non-coding regulatory sequences such as promoter and enhancer sequences. In one embodiment, particular subsets of polynucleotide sequences or genomic DNA can be used, such as, for example, particular chromosomes. Yet more particularly, the sequence of the primary polynucleotide molecules is not known. Still yet more particularly, the primary polynucleotide molecules are human genomic DNA molecules.

The nucleic acid sample can include high molecular weight material such as genomic DNA (gDNA). The sample can include low molecular weight material such as nucleic acid molecules obtained from formalin-fixed paraffin-embedded or archived DNA samples. In another embodiment, low molecular weight material includes enzymatically or mechanically fragmented DNA. The sample can include cell-free circulating DNA. In some embodiments, the sample can include nucleic acid molecules obtained from biopsies, tumors, scrapings, swabs, blood, mucus, urine, plasma, semen, hair, laser capture micro-dissections, surgical resections, and other clinical or laboratory obtained samples. In some embodiments, the sample can be an epidemiological, agricultural, forensic or pathogenic sample. In some embodiments, the sample can include nucleic acid molecules obtained from an animal such as a human or mammalian source. In another embodiment, the sample can include nucleic acid molecules obtained from a non-mammalian source such as a plant, a bacterium, a virus, or a fungus. In some embodiments, the source of the nucleic acid molecules may be an archived or extinct sample or species.

Additional non-limiting examples of sources of biological samples can include whole organisms as well as a sample obtained from a patient. The biological sample can be obtained from any biological fluid or tissue and can be in a variety of forms, including fluid, e.g., liquid or gas, tissue, e.g., solid tissue, and preserved forms of such a fluid or tissue, such as dried, frozen, and fixed forms. The sample may be of any biological tissue, cells, or fluid. Such samples include, but are not limited to, sputum, blood, serum, plasma, blood cells (e.g., white cells), ascitic fluid, urine, saliva, tears, sputum, vaginal fluid (discharge), washings obtained during a medical procedure (e.g., pelvic or other washings obtained during biopsy, endoscopy or surgery), tissue, nipple aspirate, core or fine needle biopsy samples, cell-containing body fluids, free floating nucleic acids, peritoneal fluid, and pleural fluid, or cells therefrom. Biological samples may also include sections of tissues such as frozen or fixed sections taken for histological purposes or micro-dissected cells or extracellular parts thereof. In some embodiments, the sample can be a blood sample, such as, for example, a whole blood sample. In another example, the sample is an unprocessed dried blood spot sample. In yet another example, the sample is a formalin-fixed paraffin-embedded sample. In yet another example, the sample is a saliva sample. In yet another example, the sample is a dried saliva spot sample.

Exemplary biological samples from which target nucleic acids can be derived include, for example, those from a eukaryote, for instance a mammal, such as a rodent, mouse, rat, rabbit, guinea pig, ungulate, horse, sheep, pig, goat, cow, cat, dog, primate, human or non-human primate; a plant, such as Arabidopsis thaliana, corn, sorghum, oat, wheat, rice, canola, or soybean; an algae, such as Chlamydomonas reinhardtii, a nematode such as Caenorhabditis elegans; an insect, such as Drosophila melanogaster, mosquito, fruit fly, honey bee or spider; a fish, such as zebrafish; a reptile; an amphibian, such as a frog or Xenopus laevis; a Dictyostelium discoideum; a fungi, such as Pneumocystis carinii, Takifugu rubripes, yeast, such as Saccharomyces cerevisiae or Schizosaccharomyces pombe; or protozoan, such as Plasmodium falciparum. Target nucleic acids can also be derived from a prokaryote such as a bacterium, Escherichia coli, staphylococci or Mycoplasma pneumoniae; an archaeon; a virus such as Hepatitis C virus or human immunodeficiency virus; or a viroid. Target nucleic acids can be derived from a homogeneous culture or population of organisms or alternatively from a collection of several different organisms, for example, in a community or ecosystem.

The target nucleic acids used in the methods and compositions of the present disclosure can be derived by fragmentation, such as random fragmentation. Random fragmentation refers to the fragmentation of a polynucleotide molecule from a primary nucleic acid sample in a non-ordered fashion by enzymatic, chemical, or mechanical methods. Such fragmentation methods are known in the art and use standard methods (Sambrook and Russell, Molecular Cloning, A Laboratory Manual, third edition). Moreover, random fragmentation is designed to produce fragments irrespective of the sequence identity or position of nucleotides comprising and/or surrounding the break. In one embodiment, the random fragmentation is by mechanical means such as nebulization or sonication to produce fragments of about 50 base pairs in length to about 1500 base pairs in length, still more particularly 50-700 base pairs in length, yet more particularly 50-400 base pairs in length. Most particularly, the method is used to generate smaller fragments of from 50-150 base pairs in length.

Fragmentation of polynucleotide molecules by mechanical means (nebulization, sonication, and Hydroshear, for example) results in fragments with a heterogeneous mix of blunt and 3′- and 5′-overhanging ends. It is therefore desirable to repair the fragment ends using methods or kits (such as the LUCIGEN™ DNAterminator™ End Repair Kit) known in the art to generate ends that are optimal for insertion, for example, into blunt sites of cloning vectors. In a particular embodiment, the fragment ends of the population of nucleic acids are blunt ended. More particularly, the fragment ends are blunt ended and phosphorylated. The phosphate moiety can be introduced via enzymatic treatment, for example, using polynucleotide kinase.

In a particular embodiment, the target fragment sequences are prepared with single overhanging nucleotides by, for example, activity of certain types of DNA polymerase such as Taq polymerase or Klenow exo minus polymerase which has a non-template-dependent terminal transferase activity that adds a single deoxynucleotide, for example, deoxyadenosine (A) to the 3′ ends of a DNA molecule, for example, a PCR product. Such enzymes can be used to add a single nucleotide ‘A’ to the blunt ended 3′ terminus of each strand of the double-stranded target fragments. Thus, an ‘A’ could be added to the 3′ terminus of each end repaired strand of the double-stranded target fragments by reaction with Taq or Klenow exo minus polymerase, while the universal adapter polynucleotide construct could be a T-construct with a compatible ‘T’ overhang present on the 3′ terminus of each region of double-stranded nucleic acid of the universal adapter. This end modification also prevents self-ligation of both vector and target such that there is a bias towards formation of target nucleic acids having a universal adapter at each end.

In one embodiment, fragmentation can be accomplished using a process often referred to as tagmentation. Tagmentation uses a transposome complex and combines into a single step fragmentation and ligation to add universal adapters (Gunderson et al., WO 2016/130704). A transposome complex is a transposase bound to a transposase recognition site and can insert the transposase recognition site into a target nucleic acid in a process sometimes termed “tagmentation.” In some such insertion events, one strand of the transposase recognition site may be transferred into the target nucleic acid. Such a strand is referred to as a “transferred strand.” In one embodiment, a transposome complex includes a dimeric transposase having two subunits, and two non-contiguous transposon sequences. In another embodiment, a transposase includes a dimeric transposase having two subunits, and a contiguous transposon sequence.

Some embodiments can include the use of a hyperactive Tn5 transposase and a Tn5-type transposase recognition site (Goryshin and Reznikoff, J. Biol. Chem., 273:7367 (1998)), or MuA transposase and a Mu transposase recognition site comprising R1 and R2 end sequences (Mizuuchi, K., Cell, 35:785, 1983; Savilahti, H, et al., EMBO J., 14:4893, 1995). Tn5 Mosaic End (ME) sequences can also be used by a skilled artisan.

Examples of transposon sequences useful with the methods and compositions described herein are provided in U.S. Patent Application Pub. No. 2012/0208705, U.S. Patent Application Pub. No. 2012/0208724 and Int. Patent Application Pub. No. WO 2012/061832. In some embodiments, a transposon sequence includes a first transposase recognition site and a second transposase recognition site.

Some transposome complexes useful herein include a transposase having two transposon sequences. In some such embodiments, the two transposon sequences are not linked to one another, in other words, the transposon sequences are non-contiguous with one another. Examples of such transposomes are known in the art (see, for instance, U.S. Patent Application Pub. No. 2010/0120098).

In one embodiment, tagmentation is used to produce target nucleic acids that include different universal sequences at each end. This can be accomplished by using two types of transposome complexes, where each transposome complex includes a different nucleotide sequence that is part of the transferred strand.

A population of target nucleic acids can have an average strand length that is desired or appropriate for a particular application of the methods or compositions set forth herein. For example, the average strand length can be less than about 100,000 nucleotides, 50,000 nucleotides, 10,000 nucleotides, 5,000 nucleotides, 1,000 nucleotides, 500 nucleotides, 100 nucleotides, or 50 nucleotides. Alternatively or additionally, the average strand length can be greater than about 10 nucleotides, 50 nucleotides, 100 nucleotides, 500 nucleotides, 1,000 nucleotides, 5,000 nucleotides, 10,000 nucleotides, 50,000 nucleotides, or 100,000 nucleotides. The average strand length for a population of target nucleic acids can be in a range between a maximum and minimum value set forth herein. It will be understood that amplicons generated at an amplification site (or otherwise made or used herein) can have an average strand length that is in a range between an upper and lower limit selected from those exemplified above.

In some cases, a population of target nucleic acids can be produced under conditions or otherwise configured to have a maximum length for its members. For example, the maximum length for the members that are used in one or more steps of a method set forth herein or that are present in a particular composition can be less than 100,000 nucleotides, less than 50,000 nucleotides, less than 10,000 nucleotides, less than 5,000 nucleotides, less than 1,000 nucleotides, less than 500 nucleotides, less than 100 nucleotides, or less than 50 nucleotides. Alternatively or additionally, a population of target nucleic acids can be produced under conditions or otherwise configured to have a minimum length for its members. For example, the minimum length for the members that are used in one or more steps of a method set forth herein or that are present in a particular composition can be more than 10 nucleotides, more than 50 nucleotides, more than 100 nucleotides, more than 500 nucleotides, more than 1,000 nucleotides, more than 5,000 nucleotides, more than 10,000 nucleotides, more than 50,000 nucleotides, or more than 100,000 nucleotides. The maximum and minimum strand length for target nucleic acids in a population can be in a range between a maximum and minimum value set forth above. It will be understood that amplicons generated at an amplification site (or otherwise made or used herein) can have maximum and/or minimum strand lengths in a range between the upper and lower limits exemplified above.

### Attachment of Universal Adapters

A target nucleic acid used in a method or composition described herein includes a universal adapter attached to each end. A target nucleic acid having a universal adapter at each end can be referred to as a “modified target nucleic acid.” Methods for attaching a universal adapter to each end of a target nucleic acid used in a method described herein are known to the person skilled in the art. The attachment can be through tagmentation using transposase complexes (WO 2016/130704), or through standard library preparation techniques using ligation (U.S. Pat. Pub. No. 2018/0305753).

In one embodiment, double-stranded target nucleic acids from a sample, e.g., a fragmented sample, are treated by first ligating identical universal adaptor molecules to the 5′ and 3′ ends of the double-stranded target nucleic acids. In one embodiment, the universal adapters are “matched” adapters because the two strands of the adaptors are formed by annealing complementary polynucleotide strands. In one embodiment, the universal adapters used in the method of the disclosure are referred to as “mismatched” adaptors because the adaptors include a region of sequence mismatch, i.e., they are not formed by annealing fully complementary polynucleotide strands. The general features of mismatched adaptors are further described in Gormley et al., U.S. Pat. No. 7,741,463, and Bignell et al., U.S. Pat. No. 8,053,192,). The universal adaptor typically includes universal capture binding sequences that aid in immobilizing the target nucleic acids on an array for subsequent sequencing, and universal primer binding sites useful for the sequencing.

A universal adapter can optionally include at least one index. An index can be used as a marker characteristic of the source of particular target nucleic acids on an array (U.S. Pat. No. 8,053,192). Generally, the index is a synthetic sequence of nucleotides that is part of the universal adapter which is added to the target nucleic acids as part of the library preparation step. Accordingly, an index is a nucleic acid sequence which is attached to each of the target molecules of a particular sample, the presence of which is indicative of, or is used to identify, the sample or source from which the target molecules were isolated.

Preferably an index may be up to 20 nucleotides in length, more preferably 1-10 nucleotides, and most preferably 4-6 nucleotides in length. A four nucleotide index gives a possibility of multiplexing 256 samples on the same array, a six base index enables 4096 samples to be processed on the same array.

The precise nucleotide sequence of the universal adapters is generally not material to the disclosure and may be selected by the user such that the desired sequence elements are ultimately included in the common sequences of the plurality of different modified target nucleic acids, for example, to provide for the universal capture binding sequences for immobilizing the target nucleic acids on an array for subsequent sequencing, and binding sites for particular sets of universal amplification primers and/or sequencing primers.

Additional sequence elements may be included, for example, to provide binding sites for sequencing primers which will ultimately be used in sequencing of target nucleic acids in the library, sequencing of an index, or products derived from amplification of the target nucleic acids in the library, for example on a solid support.

### Generation of Clusters

Typically, preparation of an array that includes target nucleic acids at amplification sites includes seeding the amplification sites with single-stranded nucleic acids and then amplifying the seeded target nucleic acids. In some embodiments, the method described herein includes additional steps between the annealing and the amplifying. In some other embodiments, the method described herein includes additional steps after the amplifying. Generating clonal clusters of the present disclosure also includes extension of annealed single-stranded nucleic acids to produce a complementary strand that is not methylated, resulting in a hemi-methylated double-stranded nucleic acid, and then transferring the methylated status to the complementary strand to result in both strands of each duplex being fully methylated.

The seeding of amplification sites of an array includes the use of single-stranded members of a sequencing library. The members of a sequencing library are exposed to conditions that preserve epigenetic markers, such as methylated CpG sites. Monoclonal amplification sites (amplification sites occupied with just one single-stranded member of a sequencing library) are most desirable, as sequencing monoclonal populations of amplicons yields much higher signal-to-noise ratios, increased intensity, and increased percentage of amplification sites that pass filter, all of which contribute to increased data output and data quality.

The method includes adding single-stranded members of a sequencing library to the array to result in hybridization of universal sequences on the modified target nucleic acids to capture nucleic acids present on the amplification sites. The single-stranded nucleic acids have fluidic access to the amplification sites of an array, and the single-stranded nucleic acids can be transported, for example by passive diffusion or other processes, to amplification sites. The concentration of single-stranded nucleic acids and hybridization conditions can be selected to obtain a maximum number of amplification sites occupied with one single-stranded nucleic acid. Typically, the number of the single-stranded nucleic acids added to the array exceeds the number of amplification sites in the array. In one embodiment, a concentration of single-stranded nucleic acids is added to result in a maximum of about 37% occupancy. The conditions for annealing can be isothermal or use varying temperatures. For instance, a constant temperature can be used. Alternatively, a temperature ramp can be used, typically starting at a higher temperature and reducing over time to a lower temperature. Any time period can be used, and shorter hybridization times are generally preferred. For instance, as shown in FIG. 1A, the single-strand nucleic acid 12 is shown annealed to a capture nucleic acid 11.

Hybridization of target nucleic acids to amplification sites is followed by extension of the capture nucleic acids using the hybridized seed nucleic acids as a template. Extension occurs in the presence of components that, when combined with the hybridized seed nucleic acids and the capture nucleic acids, cause the synthesis of a strand complementary to the seed nucleic acid and immobilized to the amplification site by the capture nucleic acid. The result is a plurality of amplification sites that each include one double-stranded nucleic acid. If the seed nucleic acid includes a methylated CpG site, the resulting double-stranded nucleic acid is hemi-methylated at each methylated CpG site. The extension includes a DNA polymerase, free nucleotides, and components and conditions suitable for the extension to occur. Conditions for extension of a nucleic acid at an amplification site using DNA polymerase are known to the skilled person. For instance, as shown in FIG. 1B, a double-stranded (ds) nucleic acid 14 results after the extension, where one strand made up of the capture nucleic acid 11 and the newly synthesized amplicon 13′, which is the unmethylated complement of the template strand 12. The methylated CpG dinucleotide is present on strand 12, and the unmethylated complement CpG is present on the complementary strand 13′, resulting in a hemi-methylated state for that dinucleotide present on the double-stranded nucleic acid 14.

The amplification sites are further exposed to conditions that transfer the methylation status of the seed nucleic acid to the newly synthesized complementary strand. A template-dependent DNA methyltransferase is used to transfer the methylation status. In one embodiment, the template-dependent DNA methyltransferase is the enzyme DNA (cytosine-5)-methyltransferase 1 (DNMT1). DNMT1 preferentially identifies hemi-methylated CpG sites and methylates the cytosine of the complementary unmethylated CpG dinucleotide, resulting in a CpG dinucleotide on both strands. DNA methyltransferase enzymes are commercially available, for example from Sigma Aldrich™ (catalog no. SRP0126) and from Active Motif™ (catalog no. 31404). For instance, as shown in FIG. 1C, the methylation state of the methylated CpG dinucleotide of the template strand 12 is transferred to the now methylated complementary strand 13. The amplification sites of an array can be exposed to denaturing conditions after transfer of the methylation status from the methylated seed strand to the unmethylated complementary strand. The denaturing results in amplification sites having the now methylated complementary strand covalently attached to the site by the capture nucleic acid. Denaturing conditions include, but are not limited to, formamide, heat, or alkali. In some embodiments, human DNMT1 (SEQ ID NO: 1) may be used with the methods, arrays, and compositions described herein.

The rate at which methylation occurs can be increased by increasing the concentration or amount of one or more of the active components of the reaction, for example, the concentration of enzyme. Methylation rates can also be increased in a method set forth herein by adjusting the temperature. For example, the rate of methylation of the unmethylated complementary strand can be increased by increasing the temperature up to a maximum temperature where reaction rate declines due to denaturation or other adverse events. Optimal or desired temperatures can be determined from known properties of the enzyme in use or empirically determined for a given mixture.

After the production of amplification sites containing one covalently attached single-stranded nucleic acid, the nucleic acid is amplified to generate a clonal population (also referred to as a cluster) of amplicons. In some embodiments, the amplification reaction proceeds until a sufficient number of amplicons are generated to fill the capacity of the respective amplification site. In some embodiments, the amplification is under conditions that do not preserve the methylated state of the covalently attached single-stranded nucleic acid present at an amplification site. The result is an amplification site with a plurality of amplicons. One amplicon includes the methylated CpG sites methylated by the action of the template-dependent DNA methyltransferase, such as DNMT1. The remaining amplicons include an unmethylated nucleotide sequence that is identical to, or the complement of, the original seed nucleic acid. For instance, as shown in FIG. 1D, an amplification site includes one strand 13 having methylated CpG dinucleotides and multiple copies of the same unmethylated nucleotide sequence 13′ having unmethylated CpG dinucleotides. The amplification sites also include multiple copies of the unmethylated nucleotide sequence 12′ having unmethylated CpG dinucleotides, which includes the same nucleotide sequence as strand 12 in FIGS. 1A-C but is now attached to the amplification site surface by a different capture nucleic acid 15.

In some embodiments, the amplification is under conditions that preserve the methylated states of the covalently attached single-stranded methylation state of the covalently attached single-stranded nucleic acid present at an amplification stie. Additionally, in some embodiments, a template-dependent DNA methyltransferase may be added to an amplification reaction mixture. Some template-dependent DNA methyltransferases, such as DNMT1, are not stable at high temperatures for extended periods of time. Thus, when a template-dependent DNA methyltransferase is added to an amplification mixture, it may be added following any steps performed at an elevated temperature. For example, DNMT1 may be added to an amplification reaction after denaturation of a template nucleic acid and primer annealing, such as during or after polymerase extension. In embodiments wherein an amplification reaction includes multiple steps performed at an elevated temperature, the template-dependent DNA methyltransferase may be added after each of these steps.

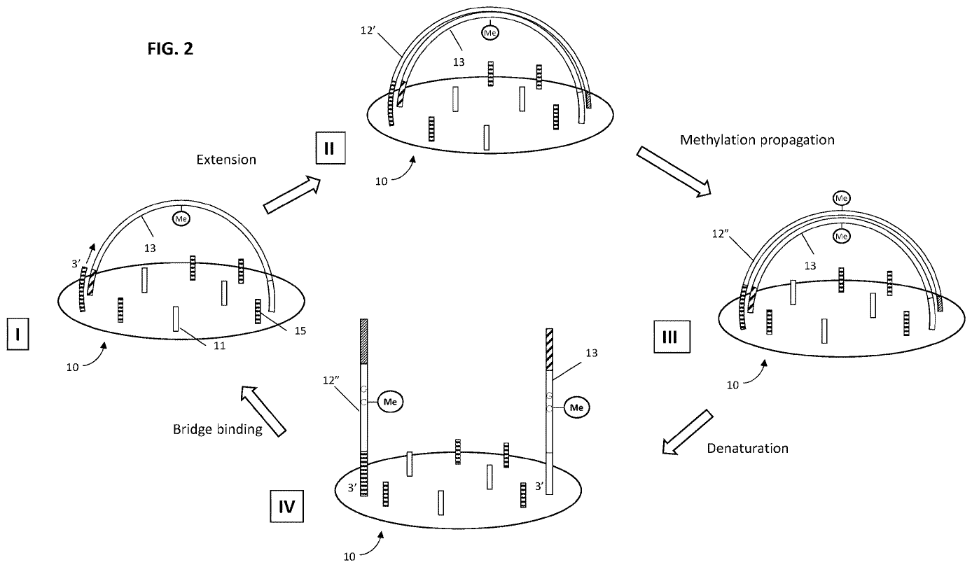

In some embodiments, amplification of one covalently attached single-stranded nucleic acid and propagation of a methylation state occur simultaneously. In some embodiments, amplification of one covalently attached single-stranded nucleic acid and propagation of a methylation state occur in alternating steps. FIG. 2 depicts an exemplary embodiment of a bridge amplification workflow wherein the bridged product is treated with a template-dependent DNA methyltransferase following extension. In this method, a single-stranded nucleic acid 13 is immobilized to an amplification site at its 5′ end. The single-stranded nucleic acid 13 is a seed nucleic acid. It is a member of a sequencing library that has been exposed to conditions that preserve an epigenetic marker present, such as the methylation state of nucleotides. The single-stranded nucleic acid 13 may be produced using, for example, a method consistent with the method depicted in FIG. 1A-C.

The amplification site additionally includes immobilized capture nucleic acids 11, 15. The seed nucleic acid has at its 3′ end a sequence that is complementary to at least part of the first immobilized capture nucleic acid 15. The seed nucleic acid has at its 5′ end a sequence that is substantially identical to at least part of the second immobilized capture nucleic acid 11. First, the single-stranded nucleic acid 13 binds to the first immobilized capture nucleic acid 15 via the sequence at its 5′ end. Next, the first immobilized capture nucleic acid 15 is extended using the single-stranded nucleic acid 13 as a template. Next, the methylation state of the single-stranded nucleic acid 13 is propagated to the newly synthesized strand 12′. The methylation state is propagated by contacting the amplification site with a template-directed DNA methyltransferase. The single-stranded nucleic acid 13 and the newly synthesized and methylated strand 12″ are denatured. Both the single-stranded nucleic acid 13 and the newly synthesized strand 12″ are attached to the amplification site at their 5′ ends. In addition, both the single-stranded nucleic acid 13 and the newly synthesized strand 12″ include complementary methylated nucleic acids, e.g., the methylation status of the single-stranded nucleic acid 13 has been propagated to the newly synthesized strand 12″.

To begin a new amplification cycle, the single-stranded nucleic acid 13 may bind a new first immobilized capture sequence 15. The newly synthesized strand 12″ may bind the second immobilized capture sequence 11. Each strand is extended to produce a new complementary strand. Following extension, the methylation state of each strand is propagated by contacting the amplification site with a template-directed DNA methyltransferase. As many DNA methyltransferases are not thermostable, this step may include adding a fresh aliquot of template-directed DNA methyltransferase to the amplification site. The process may be repeated for as many cycles as are necessary to populate the amplification site.

In some embodiments, amplification methods include, but are not limited to, solid-phase amplification. The term “solid-phase amplification” as used herein refers to any nucleic acid amplification reaction carried out on or in association with a solid support such that all or a portion of the amplified products are immobilized on the solid support as they are formed. In particular, the term encompasses solid-phase polymerase chain reaction (solid-phase PCR) and solid phase isothermal amplification which are reactions analogous to standard solution phase amplification, except that one or both of the forward and reverse amplification primers are immobilized on the solid support. Solid phase amplification includes, but is not limited to, systems such as arrays, where one primer is anchored to the surface of the array and the other is in free solution; emulsions, where one primer is anchored to a bead and the other is in free solution; and colony formation in solid phase gel matrices, where one primer is anchored to the surface and one is in free solution. In some embodiments, methods that rely on bridge amplification, where both primers are attached to a surface (see, e.g., WO 2000/018957, U.S. Pat. Nos. 7,972,820; 7,790,418 and Adessi et al., Nucleic Acids Research (2000): 28 (20): E87) are used. In some embodiments, methods are used that rely on kinetic exclusion, where recombinase-facilitated amplification and isothermal conditions amplify the library (U.S. Pat. Nos. 9,309,502, 8,895,249, 8,071,308). Methods that rely on kinetic exclusion are referred to as kinetic exclusion amplification (KEA), exclusion amplification (ExAmp), or kinetic amplification. Amplification reactions can be performed thermally or isothermally.

Kinetic exclusion can exploit a relatively slow rate for making a first copy of a target nucleic acid vs. a relatively rapid rate for making subsequent copies of the target nucleic acid or of the first copy. In some embodiments, kinetic exclusion occurs due to the relatively slow rate of target nucleic acid seeding (e.g., relatively slow diffusion or transport) vs. the relatively rapid rate at which amplification occurs to fill the site with copies of the nucleic acid seed. In another exemplary embodiment, kinetic exclusion can occur due to a delay in the formation of a first copy of a target nucleic acid that has seeded a site (e.g., delayed or slow activation) vs. the relatively rapid rate at which subsequent copies are made to fill the site. In this example, an individual site may have been seeded with several different target nucleic acids (e.g., several target nucleic acids can be present at each site prior to amplification). However, first copy formation for any given target nucleic acid can be activated randomly such that the average rate of first copy formation is relatively slow compared to the rate at which subsequent copies are generated. In this case, although an individual site may have been seeded with several different target nucleic acids, kinetic exclusion will allow only one of those target nucleic acids to be amplified. More specifically, once a first target nucleic acid has been activated for amplification, the site will rapidly fill to capacity with its copies, thereby preventing copies of a second target nucleic acid from being made at the site.

An amplification reagent can include further components that facilitate amplicon formation and in some cases, increase the rate of amplicon formation. Recombinase, such as for example UvsX, can facilitate amplicon formation by allowing repeated invasion/extension. More specifically, recombinase can facilitate invasion of a double stranded target nucleic acid by an immobilized single-stranded capture sequence and extension of the capture sequence by the polymerase using the target nucleic acid as a template for amplicon formation. This process can be repeated as a chain reaction where amplicons produced from each round of invasion/extension serve as templates in a subsequent round. The process can occur more rapidly than standard PCR since a denaturation cycle (e.g., via heating or chemical denaturation) is not required. As such, recombinase-facilitated amplification can be carried out isothermally. It is generally desirable to include ATP, or other nucleotides (or in some cases non-hydrolyzable analogs thereof) in a recombinase-facilitated amplification reagent to facilitate amplification. A mixture of recombinase and single-stranded binding (SSB) protein is particularly useful as SSB can further facilitate amplification. Exemplary formulations for recombinase-facilitated amplification include those sold commercially as TwistAmp kits by TwistDx (Cambridge, UK). Useful components of recombinase-facilitated amplification reagent and reaction conditions are set forth in U.S. Pat. Nos. 5,223,414 and 7,399,590.

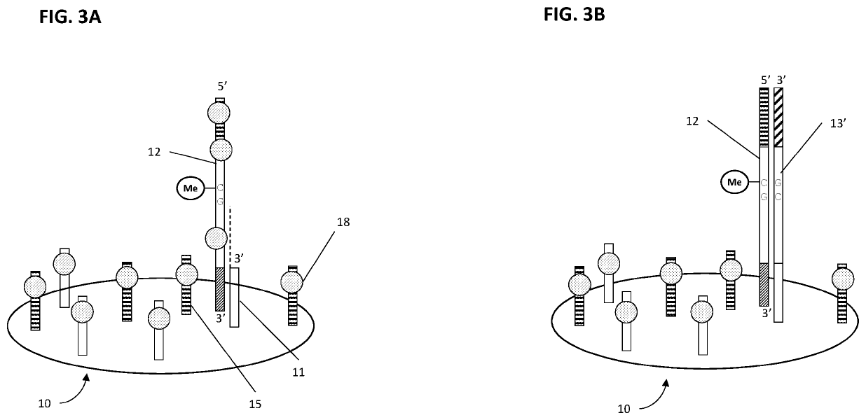

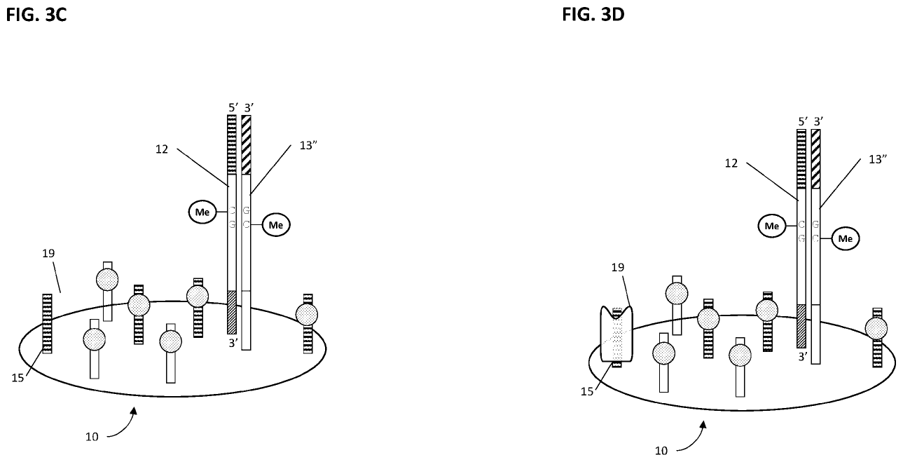

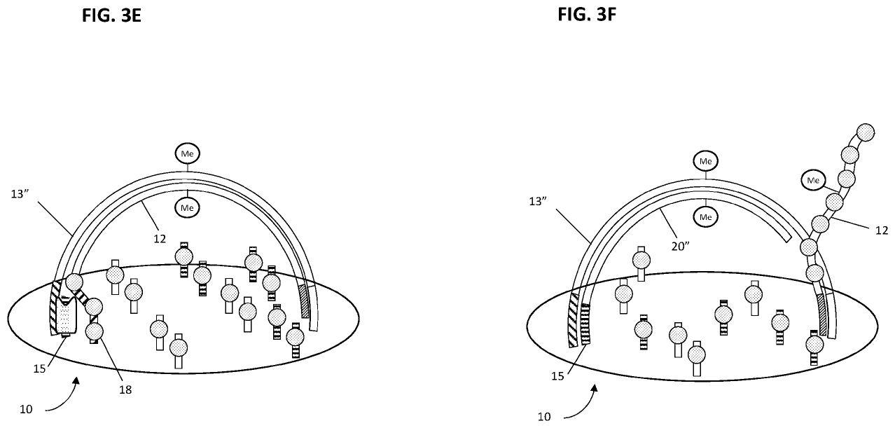

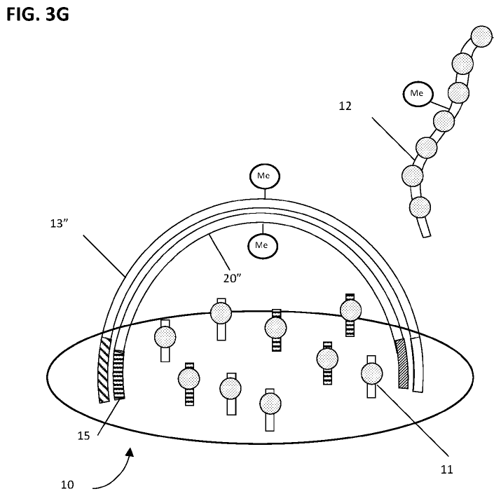

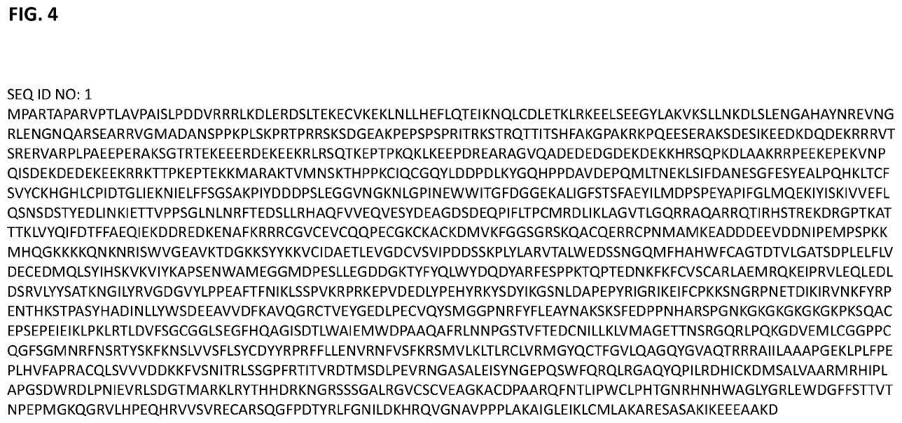

FIG. 3 depicts an exemplary embodiment of a workflow including propagation of methylation state during a protocol such as ExAmp. In the context of methylation propagation, ExAmp may be advantageous in that it does not require a heat or chemical denaturation step. As is described herein, template-dependent DNA methyltransferases are often sensitive to heat denaturation. However, such methyltransferases would likely not be denatured during an isothermal ExAmp workflow. Thus, a single initial addition of a template-dependent DNA methyltransferase may enable propagation of the methylation status of a template to an extended strand simultaneously during template amplification.

Without intending to be limited by theory, FIG. 3 depicts a method wherein a single-stranded nucleic acid 12 is hybridized to a first capture sequence 11 on an amplification site 10 (FIG. 3A). The amplification site 10 includes SSB proteins 18 that bind to the single-stranded nucleic acid and each of the capture sequences. The single-stranded nucleic acid may include at least one methylated nucleotide, such as a methylated CpG site. The first capture sequence 11 is extended by a polymerase using the single-stranded nucleic acid 12 as a template. The polymerase is able to remove the SSB proteins 18 as it processes along the single-stranded nucleic acid 12. The resulting complementary strand 13′ is immobilized to the amplification site 10 at its 5′ end (FIG. 3B).

The methylation status of the single-stranded nucleic acid 12 is propagated to the complementary strand 13′ by a template-dependent DNA methyltransferase. The template-dependent DNA methyltransferase may be added to the amplification site at this point. Alternatively, the template-dependent DNA methyltransferase may already be present at the amplification site. For example, the template-dependent DNA methyltransferase may be added simultaneously with the polymerase. The resulting product is depicted in FIG. 3C.

Next, the amplification site is treated with a recombinase 19, which can bind the second capture sequence 15 to form a nucleoprotein filament (FIG. 3D). The second capture sequence 15 can then invade the single-stranded nucleic acid 12 and complementary strand 13″ to form the structure depicted in FIG. 3E.

The second capture sequence 15 is extended using a polymerase. The polymerase displaces the single-stranded nucleic acid 12 as it processes along the complementary strand 13″ (FIG. 3F). The template-dependent DNA methyltransferase is able to act on the newly synthesized strand 20″ at any point following extension of a region complementary to a methylated nucleotide of the complementary strand 13. Thus, propagation of the methylation status to the newly synthesized strand 20″ may occur simultaneously with its extension.

Once the polymerase has reached the 5′ region of the complementary strand 13″, the single-stranded nucleic acid 12 is no longer bound to the complementary strand 13″ (FIG. 3G). The single-stranded nucleic acid 12 may be removed, leaving the resulting double-stranded, bridged structure depicted in FIG. 3G.

New strand synthesis may proceed by treating the amplification site 10 with a recombinase to facilitate invasion of the 3′ end of the newly synthesized strand 20″ with a new first capture sequence 11. Extension and propagation of methylation status may proceed to populate the amplification site 10 with a monoclonal population of methylated nucleic acids.

During or following an ExAmp-based clustering method described herein different compositions can result. For example, a composition that includes a RB69 bacteriophage UvsX, a RB69 bacteriophage single-stranded DNA binding protein (gp32), and a DNA polymerase can result. In one embodiment, the DNA polymerase is B. subtilis polymerase I (Bsu). Optionally, the composition can also include a RB69 bacteriophage UvsY. In one embodiment, the composition does not include a crowding agent, such as, but not limited to, a polyethylene glycol (PEG), dextran or Ficoll. The composition can include other components as described herein, including, but not limited to, ATP, an ATP analog, or a combination thereof. The ATP or ATP analog can be ATP, ATP-γ-S, ATP-β-S, ddATP, or a combination thereof. In one embodiment, ATP can present at a concentration of 2.3 mM to 2.8 mM and ATP-γ-S can be present at a concentration of 0.1 mM to 0.5 mM. The composition can further include one or more dNTPs, such as dATP, dGTP, dCTP, dTTP, or a combination thereof.

Another composition that can result includes ATP, ATP-γ-S, and a DNA polymerase. The ATP can be present at a concentration of 2.3 mM to 2.8 mM, and the ATP-γ-S can be present at a concentration of 0.1 mM to 0.5 mM. In one embodiment, the DNA polymerase is B. subtilis polymerase I (Bsu), E. coli DNA polymerase I Klenow fragment, B. stearothermophilus polymerase (Bst), or B. subtilis Phi-29 polymerase. The composition can further include an accessory protein such as RB69 bacteriophage UvsY, a single-stranded DNA binding protein such as RB69 bacteriophage gp32, a recombinase loading protein such as RB69 bacteriophage UvsX, or a combination thereof. The composition can optionally include a helicase such as RuvA, RuvB, or a combination thereof, and in one embodiment does not include a helicase. In one embodiment, the composition can include an ATP analog ATP-γ-S, ddATP, or a combination thereof. The composition can further include one or more dNTPs, such as dATP, dGTP, dCTP, dTTP, or a combination thereof.

A composition for amplifying nucleic acids at amplification sites, referred to herein as an “amplification reagent,” is typically capable of rapidly making copies of nucleic acids at amplification sites. An amplification reagent used in a method of the present disclosure will generally include a polymerase and nucleotide triphosphates (NTPs). Any of a variety of polymerases known in the art can be used, but in some embodiments, it may be preferable to use a polymerase that is exonuclease negative. Examples of nucleic acid polymerases suitable for use in embodiments of the present disclosure include, but are not limited to, DNA polymerase (such as Klenow fragment, T4 DNA polymerase, Bst (Bacillus stearothermophilus) polymerase), thermostable DNA polymerases (such as Taq, Vent, Deep Vent, Pfu, Tfl, and 9°N DNA polymerases) as well as their genetically modified derivatives (see, for instance, U.S. Pat. Nos. 9,677,057, 11,001,816, and U.S. Published Pat. Application U.S. Pat. Pub. No. 2020/0131484A1). In some embodiments, an amplification reagent can also include recombinase, accessory protein, and single-stranded DNA binding (SSB) protein for recombinase-facilitated amplification (see, for instance, U.S. Pat. No. 8,071,308).

The NTPs can be deoxyribonucleotide triphosphates (dNTPs) for embodiments where DNA copies are made. Typically, the four native species, dATP, dTTP, dGTP and dCTP, will be present in a DNA amplification reagent; however, analogs can be used if desired. The NTPs can be ribonucleotide triphosphates (rNTPs) for embodiments where RNA copies are made. NTPs can be modified with a fluorescent or radioactive group. A large variety of synthetically modified nucleic acids have been developed for chemical and biological methods in order to increase the detectability and/or the functional diversity of nucleic acids. These functionalized/modified molecules (e.g., nucleotide analogs) can be fully compatible with natural polymerizing enzymes, maintaining the base pairing and replication properties of the natural counterparts.

Other components of the amplification solution are added consequently to the choice of the polymerase, and they are essentially corresponding to compounds known in the art as being effective to support the activity of each polymerase. The concentration of compounds like dimethyl sulfoxide (DMSO), Bovine Serum Albumin (BSA), poly-ethylene glycol (PEG), Betaine, Triton X-100, denaturant (e.g., formamide), or MgCl₂ is well known in the prior art as being important to have an optimal amplification, and therefore the operator can easily adjust such concentrations for the methods of the present disclosure on the basis of the examples presented hereafter and the knowledge generally available.

The rate at which an amplification reaction occurs can be increased by increasing the concentration or amount of one or more of the active components of an amplification reaction, for example, the amount or concentration of polymerase, nucleotide triphosphates, or primers. In some cases, the one or more active components of an amplification reaction that are increased in amount or concentration (or otherwise manipulated in a method set forth herein) are non-nucleic acid components of the amplification reaction.

Amplification rate can also be increased in a method set forth herein by adjusting the temperature. For example, the rate of amplification at one or more amplification sites can be increased by increasing the temperature at the site(s) up to a maximum temperature where reaction rate declines due to denaturation or other adverse events. Optimal or desired temperatures can be determined from known properties of the amplification components in use or empirically for a given amplification reaction mixture. Such adjustments can be made based on a priori predictions of primer melting temperature (Tm) or empirically. In certain embodiments the temperature of an amplification reaction is at least 35° C. to no greater than 70° C. For instance, an amplification reaction can be at least 35° C. to no greater than 48° C. In contrast to other methods that determine the sequence of a nucleic acid that is anchored to a surface, the nucleic acids sequenced according to the present disclosure are attached to the surface by hybridization to a nucleic acid that is anchored to the surface. Accordingly, lower temperatures are often preferred.

Following amplification double-stranded amplicons present at the amplification sites can be converted to single-stranded amplicons by subjecting the amplicons to denaturing conditions. Denaturing conditions include, but are not limited to, formamide, heat, or alkali.

### Propagation of Methylation State

The method further includes propagating, at each amplification site, the methylated CpG dinucleotide present on one strand of the clonal population of amplicons to other unmethylated members of the clonal population. In one embodiment, an isothermal amplification reaction can be performed by incubating the amplification sites with a reaction mixture under conditions that transfer the methylated CpG dinucleotides of one strand of the clonal population to the other strands, thereby propagating the methylation status to both strands present at an amplification site. In one embodiment, the conditions include exposing the amplification sites to a template-dependent DNA methyltransferase, such as the enzyme DNMT1, and a DNA helicase or recombinase. Amplification of the methylated CpG signal occurs by many cycles of steps that include hybridizing complementary strands, transferring methylation state from one methylated CpG to the complementary unmethylated CpG, and unwinding the hybridized complementary strands. The hybridization of the complementary single-stranded nucleic acids within each amplification site results in the formation of a bridged double-stranded fragment that is hemi-methylated at each CpG. DNMT1 transfers the methylation status from a methylated CpG of one strand to the complementary unmethylated CpG of the other paired strand. The unwinding of the now fully methylated duplex can be accomplished by either strand invasion by another fragment in the cluster mediated by a recombinase or by helicase-mediated unwinding. As used herein, the term “recombinase” is intended to be consistent with its use in the art and include, for example, RecA protein, the T4 UvsX protein, the RB69 bacteriophage UvsX protein, and the like. Examples of these proteins are readily available to the skilled person (U.S. Pat. No. 8,071,308). Examples of formulations that include a helicase protein are described in U.S. Pat. Nos. 7,399,590 and 7,829,284.

A schematic example of propagation of methylation state is shown in FIG. 1E. Complementary strands 12′ and 13 shown at amplification site I anneal to form the double-stranded hemi-methylated structure shown in II. The methylation status of methylated CpG dinucleotides on strand 13 is transferred to stand 12′ to convert strand 12′ to 12 as shown in III. The double-stranded fully methylated structure shown at amplification site III is unwound and the process is repeated until the methylation status of the one methylated single-stranded nucleic acid 13 is propagated through both sub-populations of nucleic acids at the amplification sites, resulting in fully methylated clusters (amplification site IV). Although any given methylated CpG-containing strand can only undergo bridging and DNMT1 methylation with neighboring strands, sufficient density within the cluster is expected to allow the methylated CpG signal to propagate across the cluster as strands dissociate and re-anneal to different neighboring strands.

### Preparation of Clusters for Sequencing

The result of amplification and propagation of methylation state is a set of clonal single-stranded methylated amplicons at the amplification sites. The single-stranded amplicons are immobilized on the surface of an amplification site at the 5′ ends and include two populations, one population that includes a sequence that is identical to the seed nucleic acid originally used to seed the amplification site and a second population that includes a sequence complementary to the seed nucleic acid. Both populations include methylated CpG dinucleotides. For instance, see FIG. 1E, where a fully methylated cluster IV includes a population of single-stranded amplicons 12 and single-stranded amplicons 13 attached to an amplification site by capture nucleic acids 15 and 11, respectively.

Production of clusters for sequencing can include removal of one of the two populations of amplicons. Typically, one of the capture nucleic acids that is attached to the surface is cleaved to allow the optional removal one of the two populations of amplicons. The cleaving of a nucleotide sequence to permit the optional removal of a specific strand is referred to herein as “linearization.” Examples of suitable methods for linearization are described herein and are described in application number WO 2007/010251, U.S. Pat. Nos. 8,431,348, and 8,017,335.

The cleavage site is typically present in the capture nucleic acid and is typically at a pre-determined position that results in a substantial portion of the original capture nucleic acid to be retained and permit hybridization in a later step during resynthesis of the second strand as described herein. For instance, as shown in FIG. 1E, the single-stranded amplicon 12 in the cluster IV includes a cleavage site X within the capture nucleic acid 15. Cleavage results in a cleaved capture nucleic acid 15* and one population of methylated target nucleic acids, 13, as shown in FIG. 1F.

Any suitable cleavage reaction can be used to cleave at site X. Examples of cleavage reactions include, but are not limited to, enzymatic, chemical, and photochemical. Cleavage can be achieved by, for example, RNase digestion or chemical cleavage of a bond between a deoxyribonucleotide and a ribonucleotide, in which case the cleavage site can include one or more ribonucleotides; chemical reduction of a disulfide linkage with a reducing agent (e.g., TCEP), in which case the cleavage site should include an appropriate disulfide linkage; chemical cleavage of a diol linkage with periodate, in which case the cleavage site should include a diol linkage; and generation of an abasic site and subsequent hydrolysis.

Suitable cleavage techniques for use in the method of the disclosure include, but are not limited to, chemical cleavage, cleavage of an abasic site, cleavage of a ribonucleotide, photochemical cleavage, PCR stoppers, cleavage of a peptide linker, enzymatic digestion with nicking endonuclease. The person of ordinary skill in the art will recognize that use of some conditions described herein, for instance heat or alkali, may be undesirable in view of the potential for denaturation of the complementary strand from the shortened capture nucleic acid. Examples of cleavage sites and methods for cleaving the sites can be found in U.S. Pat. No. 8,765,381, and U.S. Pat. Pub. No. 2019/0309360.

In one embodiment, an abasic site is generated and cleaved. An “abasic site” is defined as a position in a nucleic acid from which the base component has been removed. Abasic sites can occur naturally in DNA under physiological conditions by hydrolysis of nucleoside residues, but can also be formed chemically under artificial conditions or by the action of enzymes. Once formed, abasic sites can be cleaved (e.g., by treatment with an endonuclease or other single-stranded cleaving enzyme, exposure to heat or alkali), providing a means for site-specific cleavage the capture nucleic acid. In one embodiment, an abasic site can be created at a pre-determined position of the capture nucleic acid and then cleaved by first incorporating deoxyuridine (U) at the pre-determined cleavage site. The enzyme uracil DNA glycosylase (UDG) can then be used to remove the uracil base, generating an abasic site. The strand including the abasic site may then be cleaved at the abasic site by treatment with endonuclease (e.g., EndoIV endonuclease, AP lyase, FPG glycosylase/AP lyase, EndoVIII glycosylase/AP lyase), heat or alkali. Abasic sites may also be generated at non-natural/modified deoxyribonucleotides other than deoxyuridine and cleaved in an analogous manner by treatment with endonuclease, heat or alkali. For example, 8-oxo-guanine can be converted to an abasic site by exposure to FPG glycosylase. Deoxyinosine can be converted to an abasic site by exposure to AlkA glycosylase. The abasic sites generated may then be cleaved, typically by treatment with a suitable endonuclease (e.g., EndoIV, AP lyase).

In one embodiment, the molecules to be cleaved may be exposed to a mixture containing the appropriate glycosylase and one or more suitable endonucleases. In such mixtures the glycosylase and the endonuclease will typically be present in an activity ratio of at least about 2:1. In a particular embodiment, the USER reagent available from New England Biolabs (NEB #M5505S) is used for the creation of a single nucleotide gap at a uracil base in a capture nucleic acid. Treatment with endonuclease enzymes gives rise to a 3′-phosphate moiety at the cleavage site, which can be removed with a suitable phosphatase such as alkaline phosphatase.

After generating clonal clusters with a single population of target nucleic acids, standard sequencing methods can be used to generate a first read of the nucleic acids. Examples of standard sequencing methods, such as sequencing-by-synthesis, are described herein. This first read generates an initial read that represents the full four-base genomic sequence prior to conversion, i.e., exposure to conditions that permit identification of the methylated residues. Thus, the initial read provides a reference sequence that is useful in the assembly of genome sequences into a consensus sequence and evaluating the sequencing after the conversion. In one embodiment, the amplification sites of the array are exposed to conditions for on-array chemical treatment to allow detection of methylated residues. On-array chemical treatment is described herein.

Sequencing of the amplicon is initiated by hybridizing a first sequencing primer to the single-stranded amplicon. Methods for sequencing are described in detail herein. In one embodiment, the first sequencing primer is complementary to a universal sequence present in the 3′ region of the amplicon. The sequencing is carried out by the sequential addition of nucleotides, in one embodiment a predetermined number of nucleotides, to the first sequencing primer using the single-stranded amplicon as the template. In some embodiments the sequencing reaction can proceed to the end of the template. For instance, as shown in FIG. 1G a sequencing primer 16 is annealed to the single-stranded amplicon 13 and is ready for extension by a DNA polymerase in a sequencing reaction.

Following sequencing, double-stranded amplicons present at the amplification sites can be converted to single-stranded amplicons by subjecting the amplicons to denaturing conditions such as formamide, heat, or alkali.

### On-Array Conversion to Identify Methylated Bases

After removal of the complementary strand produced during sequencing to re-generate the single-stranded amplicons (e.g., the single-stranded amplicon 13 in FIG. 1F), the amplification sites of the array are exposed to conditions for on-array chemical or enzymatic treatment to allow detection of methylated residues. A variety of possible conversion chemistries or other methylation detection methods can be used to identify the methylated bases. Examples include, but are not limited to, bisulfite conversion, TET-assisted pyridine borane sequencing (TAPS), and enzymatic methods employing cytosine deaminases (e.g., NEB EM-seq). Numerous enzymatic treatments may be used to identify the methylated bases, such as treatment with an APOBEC enzyme or a mutant APOBEC enzyme. APOBEC enzymes and mutants are described in more detail in U.S. Provisional Patent Application No. 63/328,444, and U.S. Provisional Patent Application No. 63/350,068, each of which is incorporated in its entirety herein by reference.

In those embodiments using sodium bisulfite conversion, methylated clusters are subjected to on-array bisulfite treatment to convert all non-methylated cytosines to uracil. For instance, as shown in FIG. 1H, treatment of the array with sodium bisulfite converts all non-methylated cytosines to uracils to result in chemically treated strand 13″.

The converted nucleic acid strands are then used in a standard paired-end resynthesis and linearization, generating a cluster of strands complementary to the reference read with G→A mutations marking unmethylated cytosines and G residues representing methylated CpG dinucleotides. For instance, FIG. 1I shows an amplification site where the strands used for the reference read have been removed and strands 12″ complementary to the reference read. Also shown is a sequencing primer 17 annealed to the single-stranded amplicon 12″.

This approach offers several advantages over standard bisulfite conversion. If insert size is approximately equal to read length, the reference read can be used to differentiate bisulfite-mediated C→T conversions from C→T SNPs, effectively allowing ‘four-base’ bisulfite sequencing where whole genome sequencing (WGS) and methylation data can be gathered in one sequencing run. Alternatively, if a longer read length is desired the conversion, e.g., bisulfite conversion, could be performed prior to the first sequencing reaction to generate paired-end reads containing the methylation signal. Another advantage is that amplification of the methylation signal prior to the conversion, e.g., bisulfite conversion, allows for more robustness to DNA damage resulting from bisulfite treatment. Bisulfite treatment can give results with single-base resolution but can create DNA damage that necessitates large amounts of sample input and results in systematic biases that make exact quantitation of methylation stoichiometry difficult. Side reactions during whole genome bisulfate chemistry steps can also result in DNA backbone cleavage, leading to dropout of regions of the genome with a high proportion of nonmethylated C residues, as well as reducing overall library conversion. Because multiple copies of the template are present and DNA damage is expected to occur stochastically, even if a large proportion of templates in the cluster are cleaved some intact templates are expected to survive bisulfite treatment. Surviving fragments can then re-populate the cluster during the resynthesis step, and the second sequencing reaction can be performed normally.

It will be appreciated bisulfite conversion is one of any number of different conversion methods that can be conducted on-flow cell after propagation of methylation with a methyltransferase enzyme. For example, an enzymatic conversion method can be used after DNMT1 propagation. One such enzymatic method is the enzymatic methyl-seq (EM-seq) method, as known to those of ordinary skill in the art, and available commercially from New England Biolabs™. EM-Seq entails oxidation of methylated cytosines by TET2 oxidase, providing protection from deamination by APOBEC in the next step. In contrast, unmodified cytosines are deaminated to uracils. The readout (detection of cytosine as evidence of methylation) is thus similar to readout of bisulfite-converted DNA.

Another such enzymatic method that can be used in combination with the methods presented herein comprises use of certain APOBEC cytidine deaminases, which have been engineered to preferentially deaminate methylated cytosine to uracil, while unmethylated cytosine remain as cytosine. Under conditions where methylated DNA is subjected to treatment with such engineered APOBEC mutants, sequence reads retain all 4 bases (A, T, C, G) greatly simplifying bioinformatic analysis including mapping sequence reads back to a reference genome. In contrast, methods such as bisulfite conversion and EM-Seq convert substantially all of the cytosines in a sample to uracil, resulting in reads which largely consist of only 3-bases (A, T, G) making bioinformatic analysis more challenging. Suitable APOBEC mutants for use in the embodiments presented herein include those described in U.S. Patent Application No. 63/328,444 filed on 7 Apr. 2022 and titled “ALTERED CYTIDINE DEAMINASES AND METHODS OF USE”, the contents of which are incorporated by reference in their entirety.

### Methods of Sequencing

The sequence of the immobilized modified target nucleic acids is determined using any suitable sequencing technique, and methods for determining the sequence of immobilized modified target nucleic acids, including strand re-synthesis, are known in the art and are described in, for instance, Bignell et al. (U.S. Pat. No. 8,053,192), Gunderson et al. (WO2016/130704), Shen et al. (U.S. Pat. No. 8,895,249), and Pipenburg et al. (U.S. Pat. No. 9,309,502).

The methods described herein can be used in conjunction with a variety of nucleic acid sequencing techniques. Particularly applicable techniques are those wherein nucleic acids are attached at fixed locations in an array such that their relative positions do not change and wherein the array is repeatedly imaged. Embodiments in which images are obtained in different color channels, for example, coinciding with different labels used to distinguish one nucleotide base type from another are particularly applicable. In some embodiments, the process to determine the nucleotide sequence of an target nucleic acid can be an automated process. Preferred embodiments include sequencing-by-synthesis (“SBS”) techniques.

SBS techniques generally involve the enzymatic extension of a nascent nucleic acid strand through the iterative addition of nucleotides against a template strand. In traditional methods of SBS, a single nucleotide monomer may be provided to a target nucleotide in the presence of a polymerase in each delivery. In some embodiments, more than one type of nucleotide monomer can be provided to a target nucleic acid in the presence of a polymerase in a delivery.

In one embodiment, a nucleotide monomer includes locked nucleic acids (LNAs) or bridged nucleic acids (BNAs). The use of LNAs or BNAs in a nucleotide monomer increases hybridization strength between a nucleotide monomer and a sequencing primer sequence present on an immobilized modified target nucleic acid.

SBS can use nucleotide monomers that have a terminator moiety or those that lack any terminator moieties. Methods using nucleotide monomers lacking terminators include, for example, pyrosequencing and sequencing using γ-phosphate-labeled nucleotides, as set forth in further detail herein. In methods using nucleotide monomers lacking terminators, the number of nucleotides added in each cycle is generally variable and dependent upon the template sequence and the mode of nucleotide delivery. For SBS techniques that use nucleotide monomers having a terminator moiety, the terminator can be effectively irreversible under the sequencing conditions used as is the case for traditional Sanger sequencing which uses dideoxynucleotides, or the terminator can be reversible as is the case for sequencing methods developed by Solexa (now Illumina, Inc.).

SBS techniques can use nucleotide monomers that have a label moiety or that lack a label moiety. Accordingly, incorporation events can be detected based on a characteristic of the label, such as fluorescence of the label; a characteristic of the nucleotide monomer such as molecular weight or charge; a byproduct of incorporation of the nucleotide, such as release of pyrophosphate; or the like. In embodiments where two or more different nucleotides are present in a sequencing reagent, the different nucleotides can be distinguishable from each other, or alternatively the two or more different labels can be the indistinguishable under the detection techniques being used. For example, the different nucleotides present in a sequencing reagent can have different labels and they can be distinguished using appropriate optics as exemplified by the sequencing methods developed by Solexa (now Illumina, Inc.).

Preferred embodiments include pyrosequencing techniques. Pyrosequencing detects the release of inorganic pyrophosphate (PPi) as particular nucleotides are incorporated into the nascent strand (Ronaghi, M., Karamohamed, S., Pettersson, B., Uhlen, M. and Nyren, P. (1996) “Real-time DNA sequencing using detection of pyrophosphate release.” Analytical Biochemistry 242 (1), 84-9; Ronaghi, M. (2001) “Pyrosequencing sheds light on DNA sequencing.” Genome Res. 11 (1), 3-11; Ronaghi, M., Uhlen, M. and Nyren, P. (1998) “A sequencing method based on real-time pyrophosphate.” Science 281 (5375), 363; U.S. Pat. Nos. 6,210,891; 6,258,568 and 6,274,320). In pyrosequencing, released PPi can be detected by being immediately converted to adenosine triphosphate (ATP) by ATP sulfurase, and the level of ATP generated is detected via luciferase-produced photons. The nucleic acids to be sequenced can be attached to features in an array and the array can be imaged to capture the chemiluminescent signals that are produced due to incorporation of a nucleotides at the features of the array. An image can be obtained after the array is treated with a particular nucleotide type (e.g., A, T, C or G). Images obtained after addition of each nucleotide type will differ with regard to which features in the array are detected. These differences in the image reflect the different sequence content of the features on the array. However, the relative locations of each feature will remain unchanged in the images. The images can be stored, processed and analyzed using the methods set forth herein. For example, images obtained after treatment of the array with each different nucleotide type can be handled in the same way as exemplified herein for images obtained from different detection channels for reversible terminator-based sequencing methods.

In another exemplary type of SBS, cycle sequencing is accomplished by stepwise addition of reversible terminator nucleotides containing, for example, a cleavable or photobleachable dye label as described, for example, in WO 04/018497 and U.S. Pat. No. 7,057,026. This approach is being commercialized by Solexa (now Illumina Inc.), and is also described in WO 91/06678 and WO 07/123,744. The availability of fluorescently-labeled terminators in which both the termination can be reversed and the fluorescent label cleaved facilitates efficient cyclic reversible termination (CRT) sequencing. Polymerases can also be co-engineered to efficiently incorporate and extend from these modified nucleotides.

In some reversible terminator-based sequencing embodiments, the labels do not substantially inhibit extension under SBS reaction conditions. However, the detection labels can be removable, for example, by cleavage or degradation. Images can be captured following incorporation of labels into arrayed nucleic acid features. In particular embodiments, each cycle involves simultaneous delivery of four different nucleotide types to the array and each nucleotide type has a spectrally distinct label. Four images can then be obtained, each using a detection channel that is selective for one of the four different labels. Alternatively, different nucleotide types can be added sequentially and an image of the array can be obtained between each addition step. In such embodiments, each image will show nucleic acid features that have incorporated nucleotides of a particular type. Different features will be present or absent in the different images due the different sequence content of each feature. However, the relative position of the features will remain unchanged in the images. Images obtained from such reversible terminator-SBS methods can be stored, processed and analyzed as set forth herein. Following the image capture step, labels can be removed and reversible terminator moieties can be removed for subsequent cycles of nucleotide addition and detection. Removal of the labels after they have been detected in a particular cycle and prior to a subsequent cycle can provide the advantage of reducing background signal and crosstalk between cycles. Examples of useful labels and removal methods are set forth herein.

In particular embodiments some or all of the nucleotide monomers can include reversible terminators. In such embodiments, reversible terminators/cleavable fluorophores can include fluorophores linked to the ribose moiety via a 3′ ester linkage (Metzker, Genome Res. 15:1767-1776 (2005)). Other approaches have separated the terminator chemistry from the cleavage of the fluorescence label (Ruparel et al., Proc Natl Acad Sci USA 102:5932-7 (2005)). Ruparel et al. described the development of reversible terminators that used a small 3′ allyl group to block extension, but could easily be deblocked by a short treatment with a palladium catalyst. The fluorophore was attached to the base via a photocleavable linker that could easily be cleaved by a 30 second exposure to long wavelength UV light. Thus, either disulfide reduction or photocleavage can be used as a cleavable linker. Another approach to reversible termination is the use of natural termination that ensues after placement of a bulky dye on a dNTP. The presence of a charged bulky dye on the dNTP can act as an effective terminator through steric and/or electrostatic hindrance. The presence of one incorporation event prevents further incorporations unless the dye is removed. Cleavage of the dye removes the fluorophore and effectively reverses the termination. Examples of modified nucleotides are also described in U.S. Pat. Nos. 7,427,673, and 7,057,026.

Additional exemplary SBS systems and methods which can be used with the methods and systems described herein are described in U.S. Pub. Nos. 2007/0166705, 2006/0188901, 2006/0240439, 2006/0281109, 2012/0270305, and 2013/0260372, U.S. Pat. No. 7,057,026, PCT Publication No. WO 05/065814, U.S. Patent Application Publication No. 2005/0100900, and PCT Publication Nos. WO 06/064199 and WO 07/010,251.

Some embodiments can use detection of four different nucleotides using fewer than four different labels. For example, SBS can be performed using methods and systems described in the incorporated materials of U.S. Pub. No. 2013/0079232. As a first example, a pair of nucleotide types can be detected at the same wavelength, but distinguished based on a difference in intensity for one member of the pair compared to the other, or based on a change to one member of the pair (e.g., via chemical modification, photochemical modification or physical modification) that causes apparent signal to appear or disappear compared to the signal detected for the other member of the pair. As a second example, three of four different nucleotide types can be detected under particular conditions while a fourth nucleotide type lacks a label that is detectable under those conditions, or is minimally detected under those conditions (e.g., minimal detection due to background fluorescence, etc.). Incorporation of the first three nucleotide types into a nucleic acid can be determined based on presence of their respective signals and incorporation of the fourth nucleotide type into the nucleic acid can be determined based on absence or minimal detection of any signal. As a third example, one nucleotide type can include label(s) that are detected in two different channels, whereas other nucleotide types are detected in no more than one of the channels. The aforementioned three exemplary configurations are not considered mutually exclusive and can be used in various combinations. An exemplary embodiment that combines all three examples, is a fluorescent-based SBS method that uses a first nucleotide type that is detected in a first channel (e.g., dATP having a label that is detected in the first channel when excited by a first excitation wavelength), a second nucleotide type that is detected in a second channel (e.g., dCTP having a label that is detected in the second channel when excited by a second excitation wavelength), a third nucleotide type that is detected in both the first and the second channel (e.g., dTTP having at least one label that is detected in both channels when excited by the first and/or second excitation wavelength) and a fourth nucleotide type that lacks a label that is not, or minimally, detected in either channel (e.g., dGTP having no label).

Further, as described in U.S. Pub. No. 2013/0079232, sequencing data can be obtained using a single channel. In such so-called one-dye sequencing approaches, the first nucleotide type is labeled but the label is removed after the first image is generated, and the second nucleotide type is labeled only after a first image is generated. The third nucleotide type retains its label in both the first and second images, and the fourth nucleotide type remains unlabeled in both images.

Some embodiments can use sequencing by ligation techniques. Such techniques use DNA ligase to incorporate oligonucleotides and identify the incorporation of such oligonucleotides. The oligonucleotides typically have different labels that are correlated with the identity of a particular nucleotide in a sequence to which the oligonucleotides hybridize. As with other SBS methods, images can be obtained following treatment of an array of nucleic acid features with the labeled sequencing reagents. Each image will show nucleic acid features that have incorporated labels of a particular type. Different features will be present or absent in the different images due the different sequence content of each feature, but the relative position of the features will remain unchanged in the images. Images obtained from ligation-based sequencing methods can be stored, processed and analyzed as set forth herein. Exemplary SBS systems and methods which can be utilized with the methods and systems described herein are described in U.S. Pat. Nos. 6,969,488, 6,172,218, and 6,306,597.

Some embodiments can use nanopore sequencing (Deamer, D. W. & Akeson, M. “Nanopores and nucleic acids: prospects for ultrarapid sequencing.” Trends Biotechnol. 18, 147-151 (2000); Deamer, D. and D. Branton, “Characterization of nucleic acids by nanopore analysis”, Acc. Chem. Res. 35:817-825 (2002); Li, J., M. Gershow, D. Stein, E. Brandin, and J. A. Golovchenko, “DNA molecules and configurations in a solid-state nanopore microscope” Nat. Mater. 2:611-615 (2003)). In such embodiments, the target nucleic acid passes through a nanopore. The nanopore can be a synthetic pore or biological membrane protein, such as a-hemolysin. As the target nucleic acid passes through the nanopore, each base-pair can be identified by measuring fluctuations in the electrical conductance of the pore. (U.S. Pat. No. 7,001,792; Soni, G. V. & Meller, “A. Progress toward ultrafast DNA sequencing using solid-state nanopores.” Clin. Chem. 53, 1996-2001 (2007); Healy, K. “Nanopore-based single-molecule DNA analysis.” Nanomed. 2, 459-481 (2007); Cockroft, S. L., Chu, J., Amorin, M. & Ghadiri, M. R. “A single-molecule nanopore device detects DNA polymerase activity with single-nucleotide resolution.” J. Am. Chem. Soc. 130, 818-820 (2008)). Data obtained from nanopore sequencing can be stored, processed and analyzed as set forth herein. In particular, the data can be treated as an image in accordance with the exemplary treatment of optical images and other images that is set forth herein.

Some embodiments can use methods involving the real-time monitoring of DNA polymerase activity. Nucleotide incorporations can be detected through fluorescence resonance energy transfer (FRET) interactions between a fluorophore-bearing polymerase and y-phosphate-labeled nucleotides as described, for example, in U.S. Pat. Nos. 7,329,492 and 7,211,414, or nucleotide incorporations can be detected with zero-mode waveguides as described, for example, in U.S. Pat. No. 7,315,019, and using fluorescent nucleotide analogs and engineered polymerases as described, for example, in U.S. Pat. No. 7,405,281 and U.S. Pub. No. 2008/0108082. The illumination can be restricted to a zeptoliter-scale volume around a surface-tethered polymerase such that incorporation of fluorescently labeled nucleotides can be observed with low background (Levene, M. J. et al. “Zero-mode waveguides for single-molecule analysis at high concentrations.” Science 299, 682-686 (2003); Lundquist, P. M. et al. “Parallel confocal detection of single molecules in real time.” Opt. Lett. 33, 1026-1028 (2008); Korlach, J. et al. “Selective aluminum passivation for targeted immobilization of single DNA polymerase molecules in zero-mode waveguide nano structures.” Proc. Natl. Acad. Sci. USA 105, 1176-1181 (2008)). Images obtained from such methods can be stored, processed and analyzed as set forth herein.

Some SBS embodiments include detection of a proton released upon incorporation of a nucleotide into an extension product. For example, sequencing based on detection of released protons can use an electrical detector and associated techniques that are commercially available from Ion Torrent (Guilford, CT, a Life Technologies subsidiary) or sequencing methods and systems described in U.S. Pub. Nos. 2009/0026082; 2009/0127589; 2010/0137143; and 2010/0282617. Methods set forth herein for amplifying target nucleic acids using kinetic exclusion amplification can be readily applied to substrates used for detecting protons. More specifically, methods set forth herein can be used to produce clonal populations of amplicons that are used to detect protons.

The above SBS methods can be advantageously carried out in multiplex formats such that multiple different target nucleic acids are manipulated simultaneously. In particular embodiments, different target nucleic acids can be treated in a common reaction vessel or on a surface of a particular substrate. This allows convenient delivery of sequencing reagents, removal of unreacted reagents and detection of incorporation events in a multiplex manner. In embodiments using surface-bound target nucleic acids, the target nucleic acids can be in an array format. In an array format, the target nucleic acids can be typically bound to a surface in a spatially distinguishable manner. The target nucleic acids can be bound by direct covalent attachment, attachment to a bead or other particle or binding to a polymerase or other molecule that is attached to the surface. The array can include a single copy of a target nucleic acid at each site (also referred to as a feature) or multiple copies having the same sequence can be present at each site or feature. Multiple copies can be produced by amplification methods such as, bridge amplification or emulsion PCR as described in further detail herein.

The methods set forth herein can use arrays having features at any of a variety of densities including, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², or higher.

An advantage of the methods set forth herein is that they provide for rapid and efficient detection of a plurality of cm², in parallel. Accordingly, the present disclosure provides integrated systems capable of preparing and detecting nucleic acids using techniques known in the art such as those exemplified herein. Thus, an integrated system of the present disclosure can include fluidic components capable of delivering amplification reagents and/or sequencing reagents to one or more immobilized target nucleic acids, the system including components such as pumps, valves, reservoirs, fluidic lines and the like. A flow cell can be configured and/or used in an integrated system for detection of target nucleic acids. Exemplary flow cells are described, for example, in U.S. Pat. Nos. 8,241,573 and 8,951,781. As exemplified for flow cells, one or more of the fluidic components of an integrated system can be used for an amplification method and for a detection method. Taking a nucleic acid sequencing embodiment as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeq™ platform (Illumina, Inc., San Diego, CA) and devices described in U.S. Pat. No. 8,951,781.

### Compositions and Articles

During practice of the methods provided by the present disclosure several compositions and/or articles of manufacture can result. For instance, a composition can result that includes a template-dependent DNA methyltransferase, such as DNMT1 enzyme and an array with a plurality of amplification sites having one double-stranded nucleic acid. One strand of the double-stranded nucleic acid is attached to the amplification site by the 5′ end and the other complementary strand is not attached to the amplification site. The double-stranded nucleic acid includes at least one hemi-methylated site; a methylated CpG dinucleotide on the strand that is not attached to the amplification site, and a complementary unmethylated CpG dinucleotide with an unmethylated CpG dinucleotide on the strand that is attached to the amplification site. In one embodiment, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the plurality of amplification sites can have the one double-stranded nucleic acid.

In another embodiment, an array can result that includes a plurality of amplification sites having one double-stranded nucleic acid. One strand of the double-stranded nucleic acid is attached to the amplification site by the 5′ end and the other complementary strand is not attached to the amplification site. The double-stranded nucleic acid includes at least one at least one methylated CpG dinucleotide on one strand and the other strand includes the complement to the at least one methylated CpG, and the cytosine of the CpG dinucleotide of each strand is methylated. The array can be part of a composition that also includes a template-dependent DNA methyltransferase, such as DNMT1 enzyme. In one embodiment, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the plurality of amplification sites can have the one double-stranded nucleic acid.

In another embodiment, an array can result that includes a plurality of amplification sites having a plurality of clonal single-stranded nucleic acids attached thereto by the 5′ end.

The plurality of single-stranded nucleic acids at each amplification site includes two populations. The first population of single-strand nucleic acids have the same nucleotide sequence and the CpG dinucleotides are not methylated. One of the strands of this population includes the methylated CpG dinucleotides. The second population of single-stranded nucleic acids are the complement of the first population, and the CpG dinucleotides are not methylated. In one embodiment, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the plurality of amplification sites can have the plurality of single-stranded nucleic acids attached thereto by the 5′ end.

In another embodiment, an array can result that includes a plurality of amplification sites where each amplification site includes a plurality of clonal single-stranded nucleic acids attached thereto by the 5′ end. The plurality of single-stranded nucleic acids at each amplification site can include two populations, a first population that is a single-stranded nucleic acid and a second population that is the complement of the single-stranded nucleic acid. The first population of the plurality of single-stranded nucleic acids includes methylated CpG dinucleotides, and the second population of the plurality of single-stranded nucleic acids includes the complement of the at least one methylated CpG dinucleotide, and the cytosine of the CpG dinucleotides of both strands is methylated. In one embodiment, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the plurality of amplification sites can have the plurality of clonal single-stranded nucleic acids immobilized thereto by the 5′ end.

In one embodiment, an array can result that includes a plurality of amplification sites, and the amplification sites each include a plurality of clonal single-stranded nucleic acids immobilized thereto by the 5′ end. The plurality of clonal single-stranded nucleic acids at each amplification site include either (i) a population that includes a template strand or (ii) a second population that includes the complement of the template strand. The plurality of clonal single-stranded nucleic acids of at least one amplification site includes methylated CpG dinucleotides. In one embodiment, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the plurality of amplification sites can have the plurality of clonal single-stranded nucleic acids immobilized thereto by the 5′ end. The array can also include a sequencing primer annealed to complementary nucleotides of the clonal single-stranded nucleic acids. The array can be part of a composition that also includes components suitable for sequencing the clonal single-stranded nucleic acids.

In a further embodiment, an array can result that includes a plurality of amplification sites that each include a plurality of clonal single-stranded nucleic acids immobilized thereto by the 5′ end. The plurality of clonal single-stranded nucleic acids at each amplification site include either (i) a population that includes a template strand, or (ii) a second population that includes the complement of the template strand, and the plurality of clonal single-stranded nucleic acids of the amplification sites includes at least one methylated CpG dinucleotide. In one embodiment, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the plurality of amplification sites can have the plurality of clonal single-stranded nucleic acids immobilized thereto by the 5′ end. In one embodiment, the nucleotides of the clonal single-stranded nucleic acids are adenine, thymine, guanine, uracil, and methylated cytosine nucleotides.

### Kits

The present disclosure also provides kits for practicing one more aspects the methods provided herein. A kit can be used for producing clonal clusters. In one embodiment, the kit can be used for on-array chemical treatment to allow detection of methylated residues. The kit can include in separate containers a template-dependent DNA methyltransferase, such as DNMT1, and a DNA helicase and/or recombinase. Optionally, the kit can include components useful for methylation detection, such as a chemical or enzymatic treatment to allow detection of methylated residues.

The components of a kit can be present in a suitable packaging material in an amount sufficient for producing at least one library. Optionally, other reagents such as a buffer solution (either prepared or present in its constituent components, where one or more of the components may be premixed or all of the components may be separate), and the like, are also included. Instructions for use of the packaged components are also typically included.

As used herein, the phrase “packaging material” refers to one or more physical structures used to house the contents of the kit. The packaging material is constructed by known methods, preferably to provide a sterile, contaminant-free environment. The packaging material has a label which indicates that the components can be used for on-array chemical treatment to allow detection of methylated residues. In addition, the packaging material contains instructions indicating how the materials within the kit are employed for practicing one more aspects of the methods provided herein. As used herein, the term “package” refers to a solid matrix or material such as glass, plastic, paper, foil, and the like, capable of holding within fixed limits one or more components of the kit. “Instructions for use” typically include a tangible expression describing the reagent concentration or at least one assay method parameter, such as the relative amounts of reagent and sample to be admixed, maintenance time periods for reagent/sample admixtures, temperature, buffer conditions, and the like.

The invention is defined in the claims. However, below there is provided a non-exhaustive listing of non-limiting exemplary aspects. Any one or more of the features of these aspects may be combined with any one or more features of another example, embodiment, or aspect described herein.

### Exemplary Aspects

Aspect 1 is an array comprising a plurality of amplification sites, wherein at least one amplification site comprises one double-stranded target nucleic acid immobilized thereto, wherein a first strand of the double-stranded target nucleic acid is attached to the amplification site at its 5′ end, wherein a second strand of the double-stranded target nucleic acid is complementary to and annealed to a region of the first strand comprising the 3′ end of the first strand, and the second strand is not attached to the amplification site, and wherein the first strand of the double-stranded target nucleic acid comprises at least one first strand CpG dinucleotide and the second strand comprises at least one complementary second strand CpG dinucleotide complementary to the at least one first strand CpG dinucleotide, wherein each cytosine of the first strand CpG dinucleotide and the complementary second strand CpG is methylated.

Aspect 2 is the composition array of aspect 1, wherein at least 10% of the plurality of amplification sites comprise a double-stranded target nucleic acid, wherein the first strand of the each double-stranded target nucleic acid comprises at least one first strand CpG dinucleotide and the second strand comprises the complementary at least one second strand CpG dinucleotide, wherein the each cytosine of the first strand CpG dinucleotide and the second strand CpG dinucleotide of each strand is methylated, and wherein the second strand of the double-stranded target nucleic acids are members of a sequencing library.

Aspect 3 is the array composition of aspect 1 or 2, further comprising a template-dependent DNA methyltransferase.

Aspect 4 is the array composition of aspect 3, wherein the template-dependent DNA methyltransferase is DNMT1 enzyme.

Aspect 5 is the array composition of any preceding aspect, further comprising components suitable for kinetic amplification or bridge amplification.

Aspect 6 is a composition comprising an array and a template-dependent DNA methyltransferase, wherein the array comprises a plurality of amplification sites comprising one double-stranded target nucleic acid immobilized to each amplification site, wherein a first strand of the double-stranded target nucleic acid is attached to the amplification site at its 5′ end, wherein a second strand of the double-stranded nucleic acid is not attached to the amplification site, is complementary to and annealed to a region of the first strand, the region comprising the 3′ end of the first strand, wherein the second strand of the double-stranded nucleic acid is a member of a sequencing library, and wherein the double-stranded target nucleic acid comprises at least one hemi-methylated CpG dinucleotide, the at least one hemi-methylated CpG having a methylated cytosine on the second strand of the double-stranded nucleic acid and an unmethylated cytosine on the first strand of the double-stranded nucleic acid.

Aspect 7 is the composition of any preceding aspect wherein the template-dependent DNA methyltransferase is DNMT1 enzyme.

Aspect 8 is an array comprising a plurality of amplification sites comprising a plurality of single-stranded nucleic acids attached thereto by the 5′ end, wherein the plurality of single-stranded nucleic acids at each amplification site comprises two populations, a first population comprising a template strand and a second population comprising the complement of the template strand, wherein one of the complements of the template strand at the amplification sites comprises at least one methylated CpG dinucleotide and the other complements of the template strand at the amplification sites do not comprise the at least one methylated CpG dinucleotide.

Aspect 9 is the array of any preceding aspect, wherein at least 10% of the plurality of amplification sites comprise the plurality of single-stranded nucleic acids at each amplification site comprising two populations.

Aspect 10 is an array comprising a plurality of amplification sites, wherein each amplification site comprises a plurality of clonal single-stranded nucleic acids attached thereto by the 5′ end, wherein the plurality of single-stranded nucleic acids at each amplification site comprises two populations, a first population comprising a first single-stranded nucleic acid and a second population comprising the complement of the first single-stranded nucleic acid, wherein each strand of the first population and each strand of the second population comprises a methylated CpG dinucleotide.

Aspect 11 is the array of any preceding aspect, wherein at least 10% of the amplification sites comprise a first and a second population of single-stranded nucleic acids, each strand of the first and second populations comprising at least one methylated CpG dinucleotide.

Aspect 12 is an array comprising a plurality of amplification sites, the amplification sites each comprising a plurality of clonal single-stranded nucleic acids immobilized thereto by the 5′ end, wherein the plurality of clonal single-stranded nucleic acids at each amplification site comprises either (i) a population comprising a template strand or (ii) a second population comprising the complement of the template strand, wherein each strand of the plurality of clonal single-stranded nucleic acids of at least one amplification site comprises at least one methylated CpG dinucleotide.

Aspect 13 is the array of any preceding aspect, further comprising a sequencing primer annealed to complementary nucleotides of the clonal single-stranded nucleic acids.

Aspect 14 is a composition comprising the array of any preceding aspect and components suitable for sequencing the clonal single-stranded nucleic acids.

Aspect 15 is an array comprising a plurality of amplification sites, the amplification sites each comprising a plurality of clonal single-stranded nucleic acids immobilized thereto by the 5′ end, wherein the plurality of clonal single-stranded nucleic acids at each amplification site comprises either (i) a population comprising a template strand or (ii) a second population comprising the complement of the template strand, wherein the plurality of clonal single-stranded nucleic acids of at least one amplification site comprises at least one methylated CpG dinucleotide, and wherein the nucleotides of the clonal single-stranded nucleic acids comprise adenine, thymine, guanine, uracil, and methylated cytosine nucleotides.

Aspect 16 is a method for producing an array comprising clonal clusters, the method comprising: providing an array comprising a plurality of amplification sites, each amplification site comprising a capture sequence and a single-stranded nucleic acid immobilized thereto, wherein the single-stranded nucleic acid is attached to the amplification site at its 5′ end, wherein a 3′ region of the single-stranded nucleic acid is complementary to and annealed to a region of the capture sequence comprising the 3′ end of the capture sequence, and wherein the single-stranded nucleic acid comprises at least one methylated CpG dinucleotide; extending the capture sequence to produce a second strand, wherein the second strand is complementary to and annealed to the single-stranded nucleic acid, and wherein the second strand comprises an unmethylated cytosine complementary to the guanine of the methylated CpG dinucleotide; and exposing the array to a template-dependent DNA methyltransferase enzyme to result in conversion of the unmethylated cytosine to a methylated cytosine, thereby converting the complementary unmethylated CpG dinucleotide of the second strand to a methylated CpG dinucleotide.

The method of aspect 16, wherein the method comprises bridge amplification.

Aspect 18 is a method for producing an array comprising clonal clusters, the method comprising providing an array comprising a plurality of amplification sites, each amplification site comprising one double-stranded nucleic acid immobilized thereto, wherein a first strand of the double-stranded nucleic acid is attached to the amplification site at its 5′ end, wherein a second strand of the double-stranded nucleic acid is a member of a sequencing library, the second strand is complementary to and annealed to a region of the first strand comprising the 3′ end of the first strand, and the second strand is not attached to the amplification site, and wherein the second strand of the double-stranded nucleic acid attached to at least one of the plurality of amplification sites comprises at least one methylated CpG dinucleotide and the first strand comprises a first unmethylated cytosine complementary to the guanine of the methylated CpG dinucleotide; and exposing the array to a template-dependent DNA methyltransferase enzyme to result in conversion of the first unmethylated cytosine to a second methylated cytosine, thereby converting the first complementary unmethylated CpG dinucleotide of the first strand to a second methylated CpG dinucleotide.

Aspect 19 is the method of aspect 18, further comprising contacting the array with a recombinase; invading the 5′ end of the second strand with a capture sequence, wherein the capture sequence is attached to the amplification site at its 5′ end; extending the capture sequence, wherein extending comprises contacting the array with a polymerase, and wherein the extended capture sequence comprises a second unmethylated CpG dinucleotide complementary to the second methylated CpG dinucleotide; and exposing the array to a template-dependent DNA methyltransferase enzyme to result in conversion of the second unmethylated cytosine to a methylated cytosine, thereby converting the second complementary unmethylated CpG dinucleotide of the extended capture strand to a third methylated CpG dinucleotide.

Aspect 20 is the method of any preceding aspect, wherein extending the capture sequence and exposing the array to a template-dependent DNA methyltransferase occur simultaneously.

Aspect 21 is the method of any preceding aspect, wherein the method comprises kinetic exclusion amplification.

Aspect 22 is the method of any preceding aspect, wherein the method comprises contacting the array with a strand-invading polymerase.

Aspect 23 is the method of any preceding aspect, wherein the method comprises contacting the array with a single-stranded DNA binding protein.

Aspect 24 is the method of any preceding aspect, wherein the method comprises contacting the array with a recombinase.

Aspect 25 is the method of any preceding aspect, wherein the template-dependent DNA methyltransferase is DNMT1 enzyme.

Aspect 26 is the method of any preceding aspect, wherein the providing comprises contacting the array comprising a plurality of amplification sites with a sequencing library comprising modified target nucleic acids, wherein each amplification site comprises two populations of capture nucleic acids, each population attached to the amplification sites at the 5′ end, wherein the modified target nucleic acids comprise a universal capture binding sequence complementary to a region of a first population of the capture nucleic acids, and wherein the contacting is under conditions suitable for hybridization between the universal capture binding sequence of a member of the sequencing library and the complementary region of the first population of the capture nucleic acids; and extending the 3′ end of the one population of capture nucleic acids by a DNA polymerase, wherein the sequence of the hybridized member of the sequencing library serves as a template strand for synthesis of a complementary strand.

Aspect 27 is the method of any preceding aspect, further comprising amplifying the first strand at each amplification site, wherein the amplifying results in each amplification site comprising the first strand and a clonal set of amplicons, wherein the first strand maintains the methylated cytosine of the at least one CpG dinucleotide, and wherein the clonal set comprises three types of nucleic acids attached to the amplification site at the 5′ end, the first strand, wherein the first strand comprises the methylated cytosine of the at least one CpG dinucleotide, a first population that comprises a nucleotide sequence that is identical to the first strand and the first population of capture nucleic acids, wherein the cytosine of the at least one CpG dinucleotide is unmethylated, and a second population that comprises the nucleotide sequence of the second strand and a second population of capture nucleic acids wherein the cytosine complementary to the at least one CpG dinucleotide is unmethylated.

Aspect 28 is the method of any preceding aspect, wherein the amplifying comprises conditions suitable for kinetic amplification or bridge amplification. Aspect 29 is the method of any preceding aspect, further comprising propagating the at least one methylated CpG dinucleotide of the first strand to the other members of the clonal set of amplicons, wherein the propagating comprises: exposing the array to a template-dependent DNA methyltransferase enzyme and an enzyme selected from a helicase, a recombinase, or a combination of both helicase and recombinase, wherein the exposing is under conditions suitable for (i) annealing of complementary strands to form bridged double-stranded nucleic acids to result in a substrate for the methyltransferase, (ii) separation of the bridged double-stranded nucleic acids, and (iii) re-annealing of different complementary strands to form bridged double-stranded nucleic acids, wherein the substrate comprises a bridged double-stranded nucleic acid comprising on one strand a methylated CpG and on the other strand the complementary unmethylated CpG dinucleotide, wherein the methyltransferase converts the complementary unmethylated CpG dinucleotide to a methylated CpG dinucleotide, and continuing the exposing until the methylated CpG dinucleotides of the first strand of the double-stranded nucleic acid is propagated to other members of the clonal population of amplicons.

Aspect 30 is a method for producing an array comprising clonal clusters, the method comprising: providing an array comprising a plurality of amplification sites, each amplification site comprising a capture sequence attached to the amplification site at its 5′ end, and a single-stranded nucleic acid immobilized to the amplification site, wherein the single-stranded nucleic acid is complementary to and annealed to a region of the capture sequence comprising the 3′ end of the capture sequence, and the single-stranded nucleic acid is not attached to the amplification site, and wherein the single-stranded nucleic acid comprises at least one methylated CpG dinucleotide, contacting the array with a single-stranded binding protein, wherein the single-stranded binding protein binds to the single-stranded nucleic acid; extending the capture sequence from its 3′ end using the single-stranded nucleic acid as a template, wherein extending comprises contacting the array with a polymerase, wherein the single-stranded nucleic acid is bound by a single-stranded binding protein as the complementary strand is synthesized, and wherein the extended capture sequence comprises a first unmethylated CpG dinucleotide complementary to the methylated CpG dinucleotide; exposing the array to a template-dependent DNA methyltransferase enzyme to result in conversion of the unmethylated cytosine to a methylated cytosine, thereby converting the first complementary unmethylated CpG dinucleotide of the extended capture sequence to a second methylated CpG dinucleotide; contacting the array with a recombinase; invading the 5′ end of the extended capture sequence with a second capture sequence, wherein the second capture sequence is attached to the amplification site at its 5′ end; extending the second capture sequence, wherein extending comprises contacting the array with a polymerase and wherein the second extended capture sequence comprises a second unmethylated CpG dinucleotide complementary to the second methylated CpG dinucleotide; and exposing the array to a template-dependent DNA methyltransferase enzyme to result in conversion of the second unmethylated cytosine to a methylated cytosine, thereby converting the second complementary unmethylated CpG dinucleotide of the first strand to a third methylated CpG dinucleotide.

Aspect 31 is a method for producing an array comprising clonal clusters, the method comprising: providing an array comprising a plurality of amplification sites, each amplification site comprising a single-stranded nucleic acid attached to the amplification site at its 5′ end, wherein the single-stranded nucleic acid comprises at least one methylated CpG dinucleotide; annealing the single-stranded nucleic acid to a capture sequence, wherein the capture sequence is complementary to a 3′ region of the single-stranded nucleic acid; extending the capture sequence from its 3′ end using the single-stranded nucleic acid as a template, wherein extending comprises contacting the array with a polymerase, and wherein the extended capture sequence comprises an unmethylated CpG dinucleotide complementary to the methylated CpG dinucleotide; and exposing the array to a template-dependent DNA methyltransferase enzyme to result in conversion of the unmethylated cytosine to a methylated cytosine, thereby converting the complementary unmethylated CpG dinucleotide of the extended capture sequence to a second methylated CpG dinucleotide; and denaturing the single-stranded nucleic acid and the extended capture sequence. Aspect 32 is the method of any preceding aspect, wherein the methylated CpG dinucleotides of the first strand are propagated to all other members of the clonal population of amplicons.

Aspect 33 is the method of any preceding aspect, wherein one of the populations of capture nucleic acids comprises a first cleavage site and the other population of capture nucleic acids comprises a second cleavage site, the method further comprising cleaving the first cleavage site to generate a cleaved population of amplicons; and removing the cleaved population of amplicons from the amplification sites to result in a non-cleaved population of amplicons immobilized to the amplification sites by the capture nucleic acids comprising the second cleavage site.

Aspect 34 is the method of any preceding aspect, further comprising hybridizing a sequencing primer to the non-cleaved population of amplicons, thereby preparing the non-cleaved population of amplicons for a first sequencing reaction.

Aspect 35 is the method of any preceding aspect, further comprising performing the first sequencing reaction to determine the sequence of at least one region of the non-cleaved population of amplicons.

Aspect 36 is the method of any preceding aspect, wherein the first sequencing reaction comprises sequencing-by-synthesis.

Aspect 37 is the method of any preceding aspect, further comprising subjecting the non-cleaved population of amplicons to conditions to generate a converted non-cleaved population of amplicons at each amplification site.

Aspect 38 is the method of any preceding aspect, wherein the conditions comprise bisulfite treatment and convert non-methylated cytosine nucleotides to uracil nucleotides.

Aspect 39 is the method of any preceding aspect, further comprising copying the non-cleaved population of amplicons at each amplification site to generate a population of complementary amplicons immobilized to the amplification sites by the capture nucleic acids comprising the first cleavage site; cleaving the second cleavage site to generate a second cleaved population of amplicons; and removing the second cleaved population of amplicons from the amplification sites to result in a second non-cleaved population of amplicons immobilized to the amplification sites.

Aspect 40 is the method of any preceding aspect, further comprising hybridizing a second sequencing primer to the second non-cleaved population of amplicons, thereby preparing the second non-cleaved population of amplicons for a second sequencing reaction.

Aspect 41 is the method of any preceding aspect, further comprising performing the second sequencing reaction to determine the sequence of at least one region of the second non-cleaved population of amplicons, wherein determining the sequence of the first and second non-cleaved populations of amplicons achieves pairwise sequencing of the first and second non-cleaved populations of amplicons.

Aspect 42 is the method of any preceding aspect, wherein the first sequencing reaction comprises sequencing-by-synthesis.

### EXAMPLES

The present disclosure is illustrated by the following examples. It is to be understood that the particular examples, materials, amounts, and procedures are to be interpreted broadly in accordance with the scope and spirit of the disclosure as set forth herein.

### Example 1

Experiments are run using NextSeq™ 2000 flow cells (ILLUMINA™). EpiScope™ Methylated HCT116 gDNA (TAKARA™ Bio catalog number 3522) is used to generate libraries and is not amplified before addition to a flow cell for cluster seeding. DNMT1 enzyme is obtained from ACTIVE MOTIF™ (catalog number 31404). USER enzyme, helicase and other enzymes are obtained from New England Biosciences™ (NEB™). EZ DNA Methylation-Direct™ Kit for bisulfite conversion is obtained from ZYMO™ (ZYMO™ catalog number D5020).

Methylated HCT116 gDNA sequencing libraries are prepared using ILLUMINA™ TruSeq™ DNA PCR-Free Library Prep kit (ILLUMINA™ catalog number 20015962) following manufacturer's instructions. Unamplified target nucleic acid libraries at a concentration of 650 picomolar (pM) are denatured using 0.1 M sodium hydroxide treatment and diluted to a final loading concentration of 130 μM in hybridization pre mix buffer (5×saline sodium citrate buffer, 0.1% tween), and loaded onto a flow cell for hybridization to the grafted primers. The hybridization procedure begins with a heating step in a stringent buffer (pre mix buffer) to ensure complete denaturation prior to hybridization. After the hybridization, which occurs during a 20 min slow cooling step from 98.5° C. down to 40.2° C., the flow cell is washed for 5 minutes with a wash buffer (0.3×saline sodium citrate buffer, 0.1% tween). The flow cell is then flushed with amplification pre-mix (2 M betaine, 20 mM Tris, 10 mM Ammonium Sulfate, 2 mM magnesium sulfate, 0.1% Triton, 1.3% DMSO, pH 8.8) at 40.2° C., followed by a first extension (FIG. 1B) in amplification mix (2 M betaine, 20 mM Tris, 10 mM Ammonium Sulfate, 2 mM magnesium sulfate, 0.1% Triton, 1.3% DMSO, pH 8.8 plus 200 μM dNTP mix and 25 units/mL of Taq polymerase (NEB™ Product ref. M0273L) at 74° C. for 90 seconds.

Following the first extension, the flow cell is washed with wash buffer (0.3×saline sodium citrate buffer, 0.1% tween) at 40.2° C. for 300 seconds. Methyltransferase mix (120 nM DNMT1 enzyme, 100 μg/mL bovine serum albumin (BSA), 160 μM S-adenosylmethionine, 50 mM Tris HCl, 1 mM EDTA, 5% glycerol, pH 7.8) is then added to the flow cell and incubated with the hemimethylated product of the first extension at 37° C. for 2 hours, resulting in methyl groups on both the original template strand and the immobilized complementary copy of the original strand (FIG. 1C).

The original template strand is denatured by heating the flow cell to 98.5° C. for 45 seconds and the flow cell is flushed with amplification mix at 98.5° C. for 10 seconds to remove the template strand from the flow cell. The immobilized strand is then amplified through 30 cycles of bridge amplification to form clusters at each site. An amplification cycle consists of annealing at 58° C. for 90 seconds, followed by extension at 74° C. for 90 seconds, followed by denaturation at 98.5° C. for 45 seconds. Once the cluster is formed (FIG. 1D), methylation is propagated from the immobilized complementary copy of the original strand to the other strands in the cluster through multiple rounds of annealing, DNMT1 transfer activity, and unwinding/denaturation (FIG. 1E-1) as described below.

After amplification of target nucleic acids at clusters and propagation of methylated CpG dinucleotides through target nucleic acids at clusters (e.g., FIG. 1E.I-E.IV) and removal of one population of strands (FIG. 1F), the first template strand of the clusters are hybridized with sequencing primer (FIG. 1G) and sequenced for 150 cycles on a NextSeq™ 2000 sequencing instrument using standard methods and reagents (ILLUMINA™).

After the sequence of the first template strands is determined, the extended sequencing primers are denatured at 98.5° C. for 45 seconds and the flow cell is flushed with wash buffer for 5 minutes to remove the template strand from the flow cell. The template strand is then treated with sodium bisulfite (CT conversion reagent solution, ZYMO™) at 64° C. for 3.5 hours, followed by washing with M-Wash Buffer (ZYMO™). Then, M-desulphonation buffer (ZYMO™) is pumped into the flow cell and incubated for 30 minutes at 20° C., followed by washing with M-Wash Buffer (ZYMO™). The result is that unmethylated cytosines of the first template strands are converted to uracil (FIG. 1H). The converted first template strands are subjected to a standard paired-end resynthesis and linearization to generate clusters having second template strands that are complementary to the first template strands. The second template strands include guanine to adenine transition mutations showing the locations of the non-methylated cytosines and guanine residues show the locations of methylated cytosines. After removal of the first template strands, the second template strands of the clusters are hybridized with sequencing primer (FIG. 1I) and sequenced for 150 cycles on a NextSeq™ 2000 sequencing instrument using standard methods and reagents (ILLUMINA™).

A comparison of the first sequence reads (e.g., those represented by FIG. 1G) with the second, paired end reads, post-bisulfite (e.g., those represented by FIG. 1I) shows the following observations. First, cytosines detected pre-bisulfite that are converted to uracil (detected as adenine on the complementary strand) indicate cytosines that were unmethylated in the original template strand. Second, cytosines detected pre-bisulfite that are not converted to uracil (detected as guanine on the complementary strand) indicate cytosines that were methylated in the original template strand. These two observations demonstrate that methylation on a target strand can be effectively propagated through the clustering process in combination with DNMT1 activity, and detected using sequencing of bisulfite-converted clusters.

### Example 2

Methylated HCT116 gDNA sequencing libraries are prepared using ILLUMINA™ TruSeq™ DNA PCR-Free Library Prep kit (ILLUMINA™ catalog number 20015962) following manufacturer's instructions. Unamplified target nucleic acid libraries at a concentration of 650 picomolar (pM) are denatured using 0.1 M sodium hydroxide treatment and diluted to a final loading concentration of 130 μM in the TwistAmp Basic Rehydration buffer and magnesium acetate reagents, and this is loaded onto the flow cell for hybridization to the grafted primers.

An Illumina NextSeq 2000 patterned flowcell, grafted with P5 and P7 primers is obtained from Illumina.

ExAmp clusters are grown using the TwistAmp Basic kit (TwistDx, Cambridge UK) as follows. A solution is prepared containing the library elements (in double stranded form) and TwistAmp Basic reagent (TwistDx, Cambridge UK). The TwistAmp Basic reagent contains an enzyme mixture that can support template dependent amplification on the surface (DNA polymerase, single-stranded binding protein and recombinase). The concentration of the library elements in solution is controlled such that the rate of hybridization capture of a library element by any feature is much lower than the rate of clonal amplification and sufficient exhaustion of the oligos available on the feature to capture another library element. The HCT116 gDNA DNA containing mixtures are used to rehydrate TwistAmp Basic freeze-dried pellets and split into two aliquots. DNMT1 enzyme and S-adenosylmethionine are added to the first mixture to a final concentration of 120 nM DNMT1 and 160 μM S-adenosylmethionine. Equivalent amounts of Tris HCl buffer are added to the second aliquot as a negative control. The aliquots are then flushed into respective lanes of the patterned flow cell at 38° C. Incubation is continued for 1 hour at 38° C. before washing with HT2 wash buffer (Illumina, Inc., San Diego Calif.)

The clusters are then linearized using standard methods and reagents (ILLUMINA™). The template strand is then treated with sodium bisulfite (CT conversion reagent solution, ZYMO™) at 64° C. for 3.5 hours, followed by washing with M-Wash Buffer (ZYMO™). Then, M-desulphonation buffer (ZYMO™) is pumped into the flow cell and incubated for 30 minutes at 20° C., followed by washing with M-Wash Buffer (ZYMO™). The result is that unmethylated cytosines of the first template strands are converted to uracil.

The template strands of the clusters are hybridized with sequencing primer and sequenced for 150 cycles on a NextSeq™ 2000 sequencing instrument using standard methods and reagents (ILLUMINA™).

Cytosines that are converted to uracil (detected as adenine on the complementary strand) indicate cytosines that were unmethylated in the original template strand. Cytosines that are not converted to uracil (detected as guanine on the complementary strand) indicate cytosines that were methylated in the original template strand. A comparison of the sequence reads from the first aliquot (e.g., those that included DNMT1 during ExAmp clustering) with the second aliquot (e.g., those that did not include DNMT1 during ExAmp clustering) shows the following observations. Cytosine is detected at CpG sites in sequence reads from the first aliquot, indicating that the presence of DNMT1 in the ExAmp mixture allowed propagation of methylation at those CpG sites during ExAmp clustering. Cytosine is not detected in sequence reads from the second aliquot, indicating that the absence of DNMT1 caused the loss of methylation information during ExAmp clustering.

These observations demonstrate that methylation on a target strand can be effectively propagated through the ExAmp clustering process in combination with DNMT1 activity, and detected using sequencing of bisulfite-converted clusters.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The disclosure is not limited to the exact details shown and described, for variations obvious to one skilled in the art will be included within the disclosure defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present disclosure. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. An array comprising a plurality of amplification sites,
wherein at least one amplification site comprises one double-stranded target nucleic acid immobilized thereto,
wherein a first strand of the double-stranded target nucleic acid is attached to the amplification site at its 5′ end,
wherein a second strand of the double-stranded target nucleic acid is complementary to and annealed to a region of the first strand comprising the 3′ end of the first strand, and the second strand is not attached to the amplification site, and
wherein the first strand of the double-stranded target nucleic acid comprises at least one first strand CpG dinucleotide and the second strand comprises at least one second strand CpG dinucleotide complementary to the at least one first strand CpG dinucleotide, wherein each cytosine of the first strand CpG dinucleotide and the second strand CpG is methylated.

2. The array of claim 1, wherein at least 10% of the plurality of amplification sites comprise a double-stranded target nucleic acid,
wherein the first strand of each double-stranded target nucleic acid comprises at least one first strand CpG dinucleotide and the second strand comprises at least one second strand CpG dinucleotide, wherein each cytosine of the first strand CpG dinucleotide and the second strand CpG dinucleotide is methylated, and
wherein the second strand of the double-stranded target nucleic acids are members of a sequencing library.

3. The array of claim 1, further comprising a template-dependent DNA methyltransferase.

4. The array of claim 3, wherein the template-dependent DNA methyltransferase is DNMT1 enzyme.

5. The array of claim 1, further comprising components suitable for kinetic amplification or bridge amplification.

6-7. (canceled)

8. An array comprising a plurality of amplification sites comprising a plurality of single-stranded nucleic acids attached thereto by the 5′ end,
wherein the plurality of single-stranded nucleic acids at each amplification site comprises two populations, a first population comprising a template strand and a second population comprising the complement of the template strand,
wherein one of the complements of the template strand at the amplification sites comprises at least one methylated CpG dinucleotide and the other complements of the template strand at the amplification sites do not comprise the at least one methylated CpG dinucleotide.

9. The array of claim 8, wherein at least 10% of the plurality of amplification sites comprise the plurality of single-stranded nucleic acids at each amplification site comprising two populations.

10-17. (canceled)

18. A method for producing an array comprising clonal clusters, the method comprising:
providing an array comprising a plurality of amplification sites, each amplification site comprising one double-stranded nucleic acid immobilized thereto,
wherein a first strand of the double-stranded nucleic acid is attached to the amplification site at its 5′ end,
wherein a second strand of the double-stranded nucleic acid is a member of a sequencing library, the second strand is complementary to and annealed to a region of the first strand comprising the 3′ end of the first strand, and the second strand is not attached to the amplification site, and
wherein the second strand of the double-stranded nucleic acid attached to at least one of the plurality of amplification sites comprises at least one methylated CpG dinucleotide and the first strand comprises a first unmethylated cytosine complementary to the guanine of the methylated CpG dinucleotide; and
exposing the array to a template-dependent DNA methyltransferase enzyme to result in conversion of the first unmethylated cytosine to a second methylated cytosine, thereby converting the first complementary unmethylated CpG dinucleotide of the first strand to a second methylated CpG dinucleotide.

19. The method of claim 18, further comprising:
contacting the array with a recombinase;
invading the 5′ end of the second strand with a capture sequence, wherein the capture sequence is attached to the amplification site at its 5′ end;
extending the capture sequence,
wherein extending comprises contacting the array with a polymerase, and
wherein the extended capture sequence comprises a second unmethylated CpG dinucleotide complementary to the second methylated CpG dinucleotide; and
exposing the array to a template-dependent DNA methyltransferase enzyme to result in conversion of the second unmethylated cytosine to a methylated cytosine, thereby converting the second complementary unmethylated CpG dinucleotide of the extended capture strand to a third methylated CpG dinucleotide.

20. The method of claim 19, wherein extending the capture sequence and exposing the array to a template-dependent DNA methyltransferase occur simultaneously.

21. The method of claim 19, wherein the method comprises kinetic exclusion amplification.

22. The method of claim 19, wherein the method comprises contacting the array with a strand-invading polymerase.

23. The method of claim 19, wherein the method comprises contacting the array with a single-stranded DNA binding protein.

24. The method of claim 19, wherein the method comprises contacting the array with a recombinase.

25. The method of claim 18, wherein the template-dependent DNA methyltransferase is DNMT1 enzyme.

26. The method of claim 18, wherein the providing comprises:
contacting the array comprising a plurality of amplification sites with a sequencing library comprising modified target nucleic acids,
wherein each amplification site comprises two populations of capture nucleic acids, each population attached to the amplification sites at the 5′ end,
wherein the modified target nucleic acids comprise a universal capture binding sequence complementary to a region of a first population of the capture nucleic acids, and
wherein the contacting is under conditions suitable for hybridization between a universal capture binding sequence of a member of the sequencing library and the complementary region of the first population of the capture nucleic acids; and extending the 3′ end of the one population of capture nucleic acids by a DNA polymerase,
wherein the sequence of the hybridized member of the sequencing library serves as a template strand for synthesis of a complementary strand.

27. The method of claim 18, further comprising amplifying the first strand at each amplification site,
wherein extending results in each amplification site comprising the first strand and a clonal set of amplicons,
wherein the first strand maintains the first methylated cytosine of the at least one CpG dinucleotide, and
wherein the clonal set comprises three types of nucleic acids attached to the amplification site at the 5′ end,
the first strand, wherein the first strand comprises the first methylated cytosine of the at least one CpG dinucleotide,
a first population that comprises a nucleotide sequence that is identical to the first strand and the first population of capture nucleic acids, wherein the cytosine of the at least one CpG dinucleotide is unmethylated, and
a second population that comprises the nucleotide sequence of the second strand and a second population of capture nucleic acids wherein the cytosine complementary to the at least one CpG dinucleotide is unmethylated.

28. The method of claim 27, wherein the amplifying comprises conditions suitable for kinetic amplification or bridge amplification.

29. The method of claim 27, further comprising propagating the at least one methylated CpG dinucleotide of the first strand to the other members of the clonal set of amplicons, wherein the propagating comprises:
exposing the array to a template-dependent DNA methyltransferase enzyme and an enzyme selected from a helicase, a recombinase, or a combination of both helicase and recombinase,
wherein the exposing is under conditions suitable for (i) annealing of complementary strands to form bridged double-stranded nucleic acids to result in a substrate for the methyltransferase, (ii) separation of the bridged double-stranded nucleic acids, and (iii) re-annealing of different complementary strands to form bridged double-stranded nucleic acids,
wherein the substrate comprises a bridged double-stranded nucleic acid comprising on one strand a methylated CpG and on the other strand the complementary unmethylated CpG dinucleotide,
wherein the methyltransferase converts the complementary unmethylated CpG dinucleotide to a methylated CpG dinucleotide, and
continuing the exposing until the methylated CpG dinucleotides of the first strand of the double-stranded nucleic acid is propagated to other members of the clonal population of amplicons.

30-42. (canceled)
